(19) United States
(12) Patent Application Publication
(10) Pub. No.: US 2025/0292865 A1
(43) Pub. Date: Sep. 18, 2025

(54) DETERMINING BIOLOGICAL PARAMETERS OF A CELL

(71) Applicant: THE UNIVERSITY OF BATH, Bath and North East Somerset (GB)

(72) Inventors: Alain NOGARET, Bath (GB); Stephen WELLS, Bath (GB); Joseph TAYLOR, Bath (GB); Paul MORRIS, Bath (GB)

(21) Appl. No.: 18/862,682

(22) PCT Filed: May 2, 2023
(86) PCT No.: PCT/GB2023/051155

§ 371 Date: Nov. 4, 2024

(30) Foreign Application Priority Data
May 3, 2022 (GB) 2206436.4

(51) Int. Cl. G16B 5/30

(52) U.S. Cl. CPC G16B 5/30

(57) ABSTRACT

The disclosure relates to a computer-implemented method for determining values of biological parameters of a cell, comprising: receiving membrane voltage readings of the cell corresponding to N time points; for a state vector comprising the biological parameters and a plurality of interdependent and time-dependent state variables, including a membrane voltage variable: setting an initial guess of the state vector and parameters; performing an optimization of the state vector by: setting the membrane voltage variable to be equal to a corresponding membrane voltage reading at a number of time points within the N time points; determining an updated value of the state vector by optimizing an objective function that operates on values of the membrane voltage variable and corresponding membrane voltage readings; and repeating the optimization with: the initial estimate of the state vector set to the updated value of the state vector; and the membrane voltage variable set to be equal to a corresponding membrane voltage reading at fewer time points within the N time points.

### FIELD

The present disclosure relates to a computer-implemented method for determining values of biological parameters of a cell, and in particular to biological parameters related to one or more ion channels of a neuron.

### BACKGROUND

Channelopathies in which certain ion channels are over-expressed or absent occur in many neurological diseases and particularly in brain cancer and Alzheimer disease (AD). Hidden mutations in ion channels at the onset of differentiation pathways result in anomalous electrical activity which is sometimes accessible to measurement but is often too subtle to be detected and diagnosed. In addition, few of the mutations that may be revealed by genetic sequencing (transcriptomics, patch-sequencing) affect electrical function. Which genes affect electrical activity, and how, is largely unknown.

Therefore, there is a need for a method capable of identifying the mutations responsible for the changes in electrical activity to develop targeted therapies and stem the decay of cognitive faculties in Alzheimer disease or brain cancer, for example.

Several hypotheses exist as to which gene might be relevant to the growth and proliferation of gliomas in brain cancer. It is known that over-expression of the stretch-activated PIEZO1 channel increases the mitosis of cancerous cells in response to tissue stiffness. Similarly, the overexpression of Kᵥ1,3 ion channels in tumour specific T-cells helps evacuate potassium ions released by intracellular necrosis which enhances tumour clearance and survival. Evidence is emerging of a positive feedback loop between glioma growth and the increased electrical excitability of the brain tissue surrounding tumours. In Alzheimer disease and related tauopathies/amyloidopathies, tangles of intraneuronal proteins cause brain lesions which modify neuronal function through (i) mechano-electrical coupling to ion channels, (ii) changes in action potential waveforms and (iii) alterations in calcium and potassium dynamics. Understanding the link between channelopathies and cell function is therefore critical if cognitive functions are to be preserved.

There are 850,000 sufferers of dementia in the UK and 11,000 brain tumours are diagnosed each year. These diseases have abysmal prognosis, hence the urgency of finding a quantitative method to diagnose and guide the development of targeted therapies. Currently there is no direct assay that links the electrical function of a cell to the underlying genetic mutations. Genetic sequencing can identify gene mutations but it cannot determine how these mutations affect function. Patch-sequencing methods can correlate, at a qualitative level, gene mutations to morphological characteristics (e.g. axonal arborisation) or the amplitude of action potentials. On the other hand, voltage clamps require multiple pharmacological manipulations and are very time consuming when the affected ion channel is unknown.

Further, clinicians cannot perform electrophysiology on healthy human brains cells due to ethical considerations and the paucity of samples. However, healthy assays are needed as a comparator to diseased cells.

Therefore, there is a need for a single shot assay that can infer the full complement of ion channels in one go and automatically output all intracellular parameters that underpin electrical waveform.

The methods, computer programs and apparatus described in the present disclosure may address one or more of the above issues.

### SUMMARY

According to a first aspect of the present disclosure there is provided a computer-implemented method for determining values of biological parameters of a cell, comprising:
receiving membrane voltage readings of the cell corresponding to N time points;
for a state vector comprising the biological parameters and a plurality of interdependent and time-dependent state variables, including a membrane voltage variable:
setting an initial guess of the state vector;
performing an optimization of the state vector by:
   setting the membrane voltage variable to be equal to a corresponding membrane voltage reading at a number of time points within the N time points;
   determining an updated value of the state vector by optimizing an objective function that operates on values of the membrane voltage variable and corresponding membrane voltage readings; and
repeating the optimization with:
   the initial guess of the state vector set to the updated value of the state vector; and
   the membrane voltage variable set to be equal to a corresponding membrane voltage reading at fewer time points within the N time points.

The cell may be a neuron. The plurality of biological parameters may relate to one or more ion channels of the neuron. The state vector may be of a conductance model of the one or more ion channels of the neuron.

The steps of repeating the optimization may be performed a plurality of times.

In a repetition of the optimization, setting the membrane voltage variable to be equal to a corresponding membrane voltage reading at fewer time points within the N time points may mean setting the membrane voltage variable at fewer time points than the membrane voltage variable was set in an immediately preceding iteration of the optimization. Depending on the number of times that the optimization has been repeated, the immediately preceding iteration may be the optimization of the state vector in which the membrane voltage variable is set to be equal to the corresponding membrane voltage reading at the number of time points within the N time points, or a subsequent repetition of the optimization.

Performing the optimization of the state vector may comprise setting the membrane voltage variable to be equal to a corresponding membrane voltage reading at intervals of M time points within the N time points. M may be less than N. Repeating the optimization may comprise using an increased value of M.

The method may comprise repeating the optimization over each time point of the time window using increasing values of M until M is equal to or greater than N.

The objective function may include a term based on a square of the difference between the membrane voltage variable and the corresponding membrane voltage for each time point.

Optimizing the objective function may comprise performing a constrained non-linear optimization over each time point to determine an updated value of the state vector that minimises the objective function.

Constraints of the constrained non-linear optimization may comprise time dependent rate equations of the state variables. The rate equations may define a relationship of a value of a state variable at a time point to values of the state vector at one or more previous or subsequent time points.

Constraints of the constrained non-linear optimisation may comprise search ranges. The search ranges may comprise an upper search boundary and a lower search boundary for each state variable and each biological parameter.

Optimising the objective function may comprise adjusting values of the plurality of biological parameters and/or state variables until the objective function satisfies a termination condition. The termination condition may comprise one of: performing a maximum number of iterations of the constrained non-linear optimization; a value of function being less than a threshold; and a gradient of the objective function being less than a threshold.

Setting the initial estimate of the state vector may comprise setting a search range for each of the state variables and each of the biological parameters. Setting the initial estimate of the state vector may comprise setting the value of each of the state variables at each time point and each of the biological parameters as a value within the corresponding search range.

The repeated optimization may be performed iteratively, a plurality of times.

The repeated optimization may result in recursive piecewise data assimilation.

The computer-implemented method may further comprise identifying one or more ion-channels of the cell affected by disease based on a comparison between the updated state vector and an expected state vector.

The computer-implemented method may further comprise simulating the effect of a medicament on the cell using the updated state vector.

The method may include identifying one or more ion-channels of the cell affected by application of the medicament based on a comparison between a state vector related to the application of the medicament and a state vector of the cell without the application of the medicament.

According to a further aspect, there is provided a data structure defining a state vector including values of biological parameters of a cell determined by the computer-implemented method described herein.

According to a further aspect, there is provided a non-transitory computer-readable storage medium comprising computer program code configured to cause one or more processors to execute the computer-implemented method described herein.

According to a further aspect, there is provided an apparatus comprising:
one or more processors; and
a memory comprising computer program code configured to cause the one or more processors to execute the computer-implemented method described herein.

The apparatus may further comprise data logging hardware for receiving membrane voltage readings.

According to a further aspect, there is provided a method for determining values of biological parameters of a cell comprising:
ex vivo, obtaining membrane voltage readings from a cell corresponding to N time points; and
using the apparatus to determine the values of biological parameters of a cell.

Also disclosed is a computer-implemented method for determining values of biological parameters of a cell, comprising:
receiving membrane voltage readings of the cell corresponding to N time points;
a state vector comprising the biological parameters and a plurality of interdependent and time-dependent state variables, including a membrane voltage variable:
setting an initial guess of the state vector and parameters;
performing an optimization of the state vector by:
   setting the membrane voltage variable to be equal to a corresponding membrane voltage reading at a number of time points within the N time points;
determining an updated value of the state vector by optimizing an objective function that operates on values of the membrane voltage variable and corresponding membrane voltage readings; and
repeating the optimization with:
the initial guess of the state vector set to the updated value of the estimated state vector; and
the membrane voltage variable set to be equal to a corresponding membrane voltage reading at fewer time points within the N time points.

Also disclosed is a method for determining a plurality of biological parameters of ion channels of a cell, comprising:
receiving membrane voltage data comprising a plurality of membrane voltages each corresponding to one of N time points of a time window;
defining a state vector of a conductance model, the state vector comprising:
a plurality of interdependent and time-dependent state variables, wherein a first time-dependent state variable represents a modelled membrane voltage; and
the plurality of biological parameters;
defining an objective function based on a square of the difference between the first time dependent state variable and the corresponding membrane voltage for each time point;
setting a starting value of the state vector;
performing recursive piecewise data assimilation by:
   performing a constrained non-linear optimization over each time point of the time window, using the starting value of the state vector, to determine an updated value of the state vector that minimises the objective function, wherein constraints of the constrained non-linear optimization comprise:
      time dependent rate equations of the plurality of state variables (defining a relationship of a value of a state variable at a time point to values of the state vector at one or more previous time points); and
      setting the first time-dependent state variable equal to the corresponding membrane voltage at intervals of every M time points of the time window, wherein M is less than N;
setting the starting value of the state vector to the updated value of the state vector; and
repeating the constrained non-linear optimization over each time point of the time window using increasing values of M until M is equal to (or greater than) N; and
outputting the plurality of biological parameters of the updated value of the state vector (from the iteration of the iteration in which M is equal to N).

There may be provided a computer program, which when run on a computer, causes the computer to configure any apparatus, including a circuit, controller, converter, or device disclosed herein or perform any method disclosed herein. The computer program may be a software implementation, and the computer may be considered as any appropriate hardware, including a digital signal processor, a microcontroller, and an implementation in read only memory (ROM), erasable programmable read only memory (EPROM) or electronically erasable programmable read only memory (EEPROM), as non-limiting examples. The software may be an assembly program.

The computer program may be provided on a computer readable medium, which may be a physical computer readable medium such as a disc or a memory device, or may be embodied as a transient signal. Such a transient signal may be a network download, including an internet download. There may be provided one or more non-transitory computer-readable storage media storing computer-executable instructions that, when executed by a computing system, causes the computing system to perform any method disclosed herein.

Throughout the present specification, the descriptors relating to relative orientation and position, such as “horizontal”, “vertical”, “top”, “bottom” and “side”, are used in the sense of the orientation of the apparatus as presented in the drawings. However, such descriptors are not intended to be in any way limiting to an intended use of the described or claimed invention.

It will be appreciated that any reference to “close to”, “before”, “shortly before”, “after” “shortly after”, “higher than”, or “lower than”, etc, can refer to the parameter in question being less than or greater than a threshold value, or between two threshold values, depending upon the context.

### BRIEF DESCRIPTION OF THE DRAWINGS

One or more embodiments will now be described by way of example only with reference to the accompanying drawings in which:

FIG. 1 illustrates a method for determining biological parameters of a cell according to an embodiment of the present disclosure;

FIG. 2 shows an example Lorenz model constructed by synchronizing a variable x to data with spurious oscillations of hidden state variables y and z;

FIG. 3 illustrates an example piecewise data assimilation fitting over three segments;

FIG. 4 illustrates an algorithm defining a generic data assimilation solver that synchronizes the neuron model to data;

FIG. 5 illustrates an algorithm for recursive piecewise data assimilation;

FIG. 6 illustrates an algorithm for recursive piecewise data assimilation including resetting an initial segment size;

FIG. 7 illustrates the convergence of a recursive piecewise data assimilation;

FIG. 8 illustrates vanishing values of the control variable and a fitting error over the assimilation window at the end of the fitting process of FIG. 7;

FIG. 9 illustrates convergence of recursive piecewise data assimilation starting from 3 successive values;

FIG. 10 illustrates model data series that was used to investigate the dependence of the biological parameters determined by piecewise data assimilation;

FIG. 11 shows an example convergence of a biological parameter as a function of iterations;

FIGS. 12A-E illustrate various profiles comparing recorded and predicted data regarding the medicament Iberiotoxin;

FIGS. 13A-F illustrate various profiles comparing recorded and predicted data for cells effected by Apamin;

FIGS. 14A-F illustrate various profiles comparing recorded and predicted data for cells effected by 4-aminopyridine (4-AP).

FIG. 15 illustrates a schematic diagram providing an overview of stages according to some aspects of the invention and their implementation;

FIG. 16 illustrates a schematic block diagram of a system which may be used to implement the methods described previously; and

FIG. 17 discloses a computer readable storage medium.

### DETAILED DESCRIPTION

A significant challenge in estimating the parameters of biological models is the difficulty of finding a unique set of parameters that match model predictions to data measurements over a time window. Inferring the true set of neuron-based model parameters belongs to the class of P vs NP complete problems. It is critical that a single-valued set of parameters be obtained if the method is to provide relevant information about the biology. Unfortunately state of the art inference methods infer both actual AND spurious solutions associated with local minima. The ability of these methods to reliably estimate (i) a unique set of parameters and (ii) demonstrate their relevance to biology has never been done. These are the disruptive steps brought by methods according to the present disclosure which now make it relevant as an assay. In some embodiments, the new computational methods can estimate ionic currents and parameters with sufficient accuracy to determine pharmacologically induced charges in ion channel conduction thus validating the relevance of predictions to biology.

State of the art in data assimilation has so far focused on building predictive neuron/network models. Models with wrong parameters (associated with local minima) could still accurately predict the state of a neuron. Because these parameters lacked the necessary accuracy to describe changes in the underlying biology, they could not until now be used as diagnosis tool evaluating the effect of drug or disease. Examples of papers focusing of predictive models of songbird neurons, rodent CA1 and respiratory neurons and neuromorphic neurons can be found in the literature. None of the prior art works claim relevance of their estimated parameters to biology—only the time dependence of the membrane voltage.

Biological models often have a large number of parameters. Subsidiary challenges include:
Models are non-linear which in practice means that the model can switch to a completely different state if one tries to linearly extrapolate parameters to fit data.
Data contain noise and experimental error that introduce uncertainty in the parameter estimates.
Models are (good) approximations of the biological reality with the effect that lack of knowledge on the exact model introduces error in the parameter estimates.
Observability: only a few of the state variables of the model can be measured. For example, the membrane voltage or calcium concentration can be recorded and used to infer information on the model but the state of gate variables of individual ion channels is inaccessible to measurement. This implies that information about the state of an ionic gate needs to be inferred indirectly from membrane voltage observations. This inference does not always yield a unique value for gate variables in which case the system is not observable. In other words, the data do not contain enough information for the model to be uniquely determined.
Identifiability: this is a related issue concerning the criteria for choosing the stimulation protocol. In order to constrain parameters in a unique way, the current waveform must carry sufficient information to induce voltage oscillations in a range controlled by all parameters. For example, a protocol that never induces action potentials will not allow the biological neuron to generate information on parameters that control depolarisation. For this current protocol the model will be over-specified, with the effect that multiple sets of parameters will be equally good at predicting sub-threshold oscillations.

The present disclosure provides methods and apparatus with which biological parameters can be uniquely identified. Examples are provided for two representative multichannel conductance models—the RVLM (Rostro ventro lateral medulla) model and the hippocampal neuron model.

Example methods of the present disclosure provide a piecewise data assimilation method that can extract the biological parameters including ionic conductances, gate recovery times and activation thresholds across the full complement of ion channels by analysing the membrane voltage oscillations of an electrically active cell. The parameter solution is single valued and has been found to have the accuracy to detect disease induced mutations in ion channel proteins or pharmacologically induced changes.

The piecewise data assimilation method can select the biologically meaningful solution among all others. The method can account for an eventual lack of observability by discarding all non-physical solutions of the inference problem and iteratively increasing accuracy as the bias towards the biological solution is removed. As disclosed below, piecewise data assimilation can achieve a 100% convergence probability and estimate all ˜70 parameters with an accuracy better than 0.1%. In some examples, identifiability criteria are met by using current waveforms that cover the full dynamic range of the neuron. As explained below, optimal accuracy on all parameters can advantageously be obtained independently of the choice of assimilation widow. Nonlinear conductance models can be easily inferred using interior point optimization. Residual data noise and residual model error up to a threshold level can be well tolerated by piecewise data assimilation thanks to statistical sampling over the length of the assimilation window. In order to increase the accuracy on parameter solution, it is desirable to sample data over a longer time window without increasing the number of data points. To this end some examples implement a smart sampling method that uses an adaptive step size within piecewise data assimilation to sample action potentials with a greater resolution than subthreshold oscillations. The latter determine fewer parameters in the model.

The disclosed approach is sufficiently accurate to identify ion channels which have been inhibited pharmacologically and to quantify the potency and selectivity of inhibitors, in good agreement with IC50. This makes the approach suitable for identifying disease induced mutations in ion channel proteins. The obtained models are also relevant to diagnose channelopathies due to disease (Alzheimer neurons or tumoral cells) and infer mutations in specific ion channels induced by these diseases. The present method is also relevant to quantifying the effect of drugs on diseased ion channels.

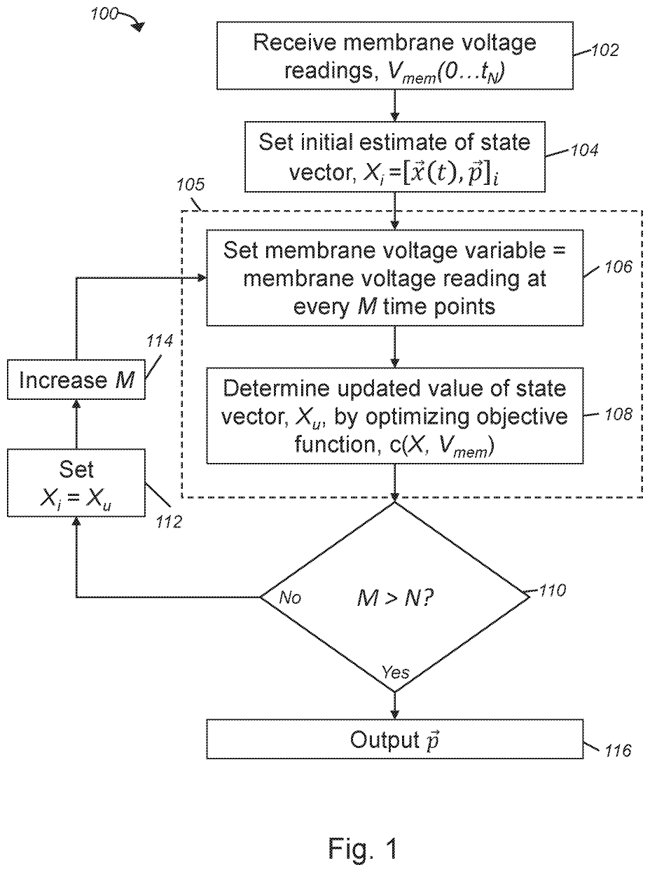

FIG. 1 illustrates a method 100 for determining biological parameters of a cell according to an embodiment of the present disclosure. In some examples, the cell may be a neuron and the biological parameters may relate to one or more ion channels of the neuron.

A first step 102 comprises receiving membrane voltage readings, Vₘₑₘ(0 . . . t_{N}), of the cell corresponding to a series of readings taken at N time points.

A second step 104 comprises setting an initial estimate of a state vector, Xᵢ=[\vec{x}ᵢ(t),\vec{p}]. The state vector comprises a plurality of interdependent and time-dependent state variables, \vec{x}(t)=x₁(t), . . . x_{L}(t), including a membrane voltage variable, x₁(t). The remaining state variables, x₂(t), . . . x_{L}(t), may be referred to as the gate variables of a conductance model. The state vector also comprises the time independent biological parameters, \vec{p}=X_{L+1}, . . . , X_{L+P}.

Setting an initial guess (which may also be referred to as an estimate, although typically the term “estimate” is used herein to refer to an output whereas the “guess” is an input) of the state vector may comprise setting a search range for each of the state variables and the biological parameters and setting the initial guess as a value (such as the mid-point) within the search range. As explained below, the exact value may not matter because the method converges to a unique solution of the state vector over the assimilation window. The bounds of the search ranges may be guided by biological considerations, as explained further below in relation to table 2. In some examples, setting the initial guess may comprise setting an initial guess for each time dependent state variable at each time point across the assimilation window. In this example, the conductance model defines a relationship between a value of a state variable at a time point to values of the state vector at one or more previous time points. Example conductance models of ion channels of neurons are described in detail below.

The method proceeds by performing 105 an optimisation. A first sub-step 106 of performing 105 the optimisation comprises setting the membrane voltage variable, x₁(t), equal to a corresponding membrane voltage reading, Vₘₑₘ(t), at a number of time points within the N time points. In this example, the membrane voltage variable is set equal to the corresponding membrane voltage reading at every M time points, where M<N. In a first iteration of the optimisation 105, an initial value of M=M₀ may be used. In some examples, M₀ may be set prior to, after or at the same time as setting the initial estimate of the state vector. In some examples, M₀ may be any integer greater than or equal to 2.

A second sub-step 108 of performing 105 the optimisation comprises determining an updated value of the state vector, Xᵤ, by optimising an objective function, c(X, Vₘₑₘ). The objective function operates on values of the membrane voltage variable, X, and the corresponding membrane voltage readings, Vₘₑₘ. For example, the objective function may be based on a sum of squares of the difference between the membrane voltage variable and the corresponding membrane voltage reading. Determining an updated value of the state vector, Xᵤ, may comprise performing a constrained non-linear optimization over each time point by adjusting a value of the state vector until a value of the objective function satisfies a termination criterion. The optimization may comprise a gradient descent algorithm or other known optimization algorithms. In some examples, the optimization may comprise gradient descent with interior point optimization code (IPOPT) incorporating the MA97 linear solver for sparse differential equations (HSL). The termination criterion may comprise one of: performing a maximum number of iterations of the constrained non-linear optimization; a value of function being less than a threshold; a gradient of the objective function being less than a threshold; or any other known optimisation termination criterion. Constraints of the non-linear optimisation comprise: (i) the setting of the membrane voltage variable equal to the membrane voltage reading every M time points; (ii) time dependent rate equations of the state variables, wherein the state variables define a relationship of a value of a state variable at a time point to values of the state vector at one or more previous time points; and (iii) the search ranges of each of the state variables and the biological parameters. The time-dependent rate equations may be of a conductance model of one or more ion channels of a cell, such as a neuron. Further details on performing the optimisation are provided below.

In some examples, the state vector may comprise two further time dependent variables—a regularization term (or control variable) and a time derivative of the regularization term. The objective function may further comprise the regularisation term, for example a square of the regularization term. The regularization term can stabilize convergence during data assimilation through the introduction of a conditional Lyapunov exponent. The term can vanish as the state approaches the minimum of the objective function.

The method 100 further comprises repeating the optimisation with: (i) the initial estimate of the state vector, Xᵢ, set to the updated value of the state vector, Xᵤ; and (ii) the membrane voltage variable set to be equal to a corresponding membrane voltage reading at fewer time points within the N time points (i.e. an increased value of M). Repeating the optimisation may be performed iteratively, a plurality of times. In this example, the method repeats until M=N by including a step of determining 110 whether M is greater than N. If M is not greater than N, the method proceeds to step 112 and sets the initial estimate of the state vector, Xi, equal to the updated value of the state vector, Xᵤ, from the previous iteration. The method then proceeds to step 114 and increases M, for example by incrementing I. The method then proceeds to repeat step 105. If at step 110, M is greater than N, then the method terminates by outputting 116 the biological parameters, \vec{p}. In some examples, the biological parameters may be output as part of the latest updated value of the state vector, Xᵤ.

The method of constraining the membrane voltage variable to be equal to the membrane voltage reading every M steps may be referred to as piecewise data assimilation. Repeating the piecewise data assimilation with increasing values of M may be referred to as recursive piecewise data assimilation. It has been found that the use of recursive piecewise data assimilation allows a selection of a biologically meaningful solutions from other potential optimization solutions occurring at local minima with excellent accuracy. The method accounts for the eventual lack of observability of the state variables by discarding all non-physical solutions of the inference problem (when M=M₀) and iteratively increasing its accuracy as the bias towards the biological solution is removed (M tends to N). Remarkably, piecewise data assimilation achieves a 100% convergence probability and estimates all ˜70 parameters of an ion channel model with an accuracy better than 0.1%.

In this way, aspects of the computational method can be considered to implement recursive piecewise data assimilation to estimate a unique set of parameters by synchronizing neuron-based conductance to electrophysiological recordings. In some example embodiments, the method is able to determine biological parameters with ˜100% success rate and accuracy of better than 0.1%. These parameters constitute the fingerprints of electrically active ion channels, and the genes coding them.

The computational method may be used to implement a single shot assay capable of identifying the ion channels affected by disease (channelopathies) and quantifying the changes (ion conductances, voltage thresholds and recovery time constants) caused by disease. The assay has demonstrated the ability to estimate the potency and selectivity of inhibitor drugs on the ion channels of CA1 neurons, as discussed further with regards to the specific examples below.

In addition, the state vector that is generated by a completed model obtained by the method can be considered to provide a “digital twin” of the biological neuron, or other cell, that it represents. The digital twin can be used to simulate the effect of drugs on virtual diseased neurons, optimising drug design, and comparing diseased neurons to virtual healthy neurons when the latter cannot by obtained from human patients.

The following provides a detailed description of specific examples of methods according to the present disclosure.

### 1. Mathematical and Computational Method

### 1.1. Primal-Dual Optimization

In some examples, the state of the neuron is represented by a state vector with L+P+2 vector components:
The first L vector components are the state variables of a conductance model: the membrane voltage variable x₁(t) plus the gate variables x₂(t), . . . , x_{L}(t).
The next K vector components are the conductance model parameters: \vec{p}=x_{L+1}, . . . , x_{L+P}. These are the biological parameters sought. Model parameters are effectively time independent state variables (\dot{x}_{L}+1=0, . . . , \dot{x}_{L+P}=0). They include the ion channel conductances, gate recovery times, activation/inactivation voltage thresholds, soma area, and membrane capacitance.
The next two variables are the regularization term u(t) and its time derivative du(t).

The optimization of the state vector obtains the vector X(tᵢ) at each point i∈[0,N] of the assimilation window of duration T. When the mesh is evenly spaced tᵢ=iT/N.

In some examples, the objective function comprises a least-square cost function that measures the distance between the membrane voltage variable x₁(t) and the measured membrane voltage Vₘₑₘ(t) across the assimilation window:

$$c ( \vec{x} ( 0 ) , \vec{p} ) = \frac{1}{2} {\sum }_{i = 0}^{i = N} [ {( {x}_{1} ( {t}_{i} , \vec{x} ( 0 ) ) - {V}_{mem} ( {t}_{i} ) )}^{2} + {u}^{2} ( {t}_{i} ) ] \qquad ( 1 )$$

The regularization term u(tᵢ)>0 may assist to stabilize convergence during data assimilation through the introduction of a conditional Lyapunov exponent. This regularization term vanishes as the state approaches the minimum of the cost function.

The rate equations of motion for the state vector are defined as:

$$\frac{{dx}_{1} ( t )}{dt} = {F}_{1} [ \vec{x} ( t ) , \vec{x} ( 0 ) ] - u ( t ) ( {x}_{1} ( t ) - {V}_{mem} ( t ) ) \qquad ( 2 )$$

$$…$$

$$\frac{{dx}_{l} ( t )}{dt} = {F}_{l} [ \vec{x} ( t ) , \vec{x} ( 0 ) ] , l = 2 , … , L$$

The functions F₁, . . . , F_{L} are specified by the conductance model of the cell:

$$\frac{{dx}_{1} ( t )}{dt} = \frac{1}{C} \{ - {\sum }_{ions} {J}_{ions} ( t ) + {I}_{inj} ( t ) / A \} \qquad ( 3 )$$

$$…$$

$$\frac{{dx}_{l} ( t )}{dt} = \frac{{x}_{l ∞} ( {x}_{1} ) - {x}_{l} ( t )}{{τ}_{l} ( {x}_{1} )} , l = 2 … L$$

with ionic currents of the form:

$${J}_{ion} ( t ) = {\overline{g}}_{ion} {x}_{i}^{a} {x}_{i + 1}^{b} ( {E}_{ion} - {x}_{1} ( t ) ) \qquad ( 4 )$$

where a,b∈N, I∈[2, . . . , L]. Eᵢₒₙ and g̅ᵢₒₙ are the reversal potential and the maximum conductance of the ionic species respectively. A is the surface area of the soma through which the current protocol is injected Iᵢₙⱼ(t). The sigmoid activation curve and the recovery constant both depend on the membrane voltage through:

$${x}_{l , ∞} ( {x}_{1} ) = \frac{1}{2} [ 1 + tan h ( \frac{{x}_{1} - {V}_{th , l}}{δ {V}_{l}} ) ] \qquad ( 5 )$$

$${τ}_{l} ( {x}_{1} ) = {t}_{l} + {ϵ}_{l} [ 1 - tan {h}^{2} ( \frac{{x}_{1} - {V}_{th , l}}{δ {V}_{t , l}} ) ] \qquad ( 6 )$$

Example proof-of-concept results in sections 2 and 3 are based on the RVLM and the hippocampal CA1 neuron models which are representative of most classes of neurons in the central nervous system. These models differ in the combination of ion channels whose equations (Eq. 4) are tabulated in public literature, for example Taylor J D, Winnall S, Nogaret A, Estimation of neuron parameters from imperfect observations. PLoS Comput. Biol., 16 Jul. 2020, 16(7): e1008053. https://doi.org/10.1371/journal.pcbi.1008053; and Nogaret A, Meliza C. D., Margoliash D., Abarbanel H. D. I., “Automatic Construction of Predictive Neuron Models through Large Scale Assimilation of Electrophysiological Data”, Scientific Reports, 8 Sep. 2016, 6:32749, DOI: 10.1038/srep32749.

The RVLM model can be written as:

$$C \frac{dV}{dt} = - {J}_{NaT} - {J}_{K} - {J}_{CaT} - {J}_{HCN} - {J}_{L} - {I}_{inj} ( t ) / A \qquad ( 7 )$$

Similarly, the Hippocampal CA1 model can be written as:

$$C \frac{dV}{dt} = - {J}_{NaP} - {J}_{NaT} - {J}_{K} - {J}_{A} - {J}_{M} - {J}_{HCN} - \frac{{I}_{inj} ( t )}{A} \qquad ( 8 )$$

where the values of the various parameters of equations 7 and 8 are as defined in table 1.

TABLE 1

| | |
|---|---|
| Transient | J_{Nat} = g_{NaT}m³h (E_{Na} − V) |
| sodium | |
| Non activating | J_{K} = g_{K}n⁴(E_{K} − V) |
| potassium | |
| Hyper- | J_{HCN} = g_{HCN}^{r} (E_{HCN} − V) |
| polarised | |
| activated | |
| cation | |
| Low threshold calcium | ${J}_{CaT} = {g}_{CaT} {p}^{2} {qz}^{2} \frac{{VF}^{2}}{RT} \frac{{[ {Ca}^{2 +} ]}_{i} - {[ {Ca}^{2 +} ]}_{o} exp ( - zFV / RT )}{1 - exp ( zFV / RT )}$ |
| Persistent | J_{NaP} = g_{Nap}m(E_{Na} − V) |
| sodium | |
| Rapidly | J_{A} = g_{A}m⁴h(E_{K} − V) |
| inactivating | |
| potassium | |
| Muscarinic | J_{M} = m(E_{K} − V) |
| sensitive | |
| potassium | |

The m,n are activation state variables and the h are inactivation variables. Their generic voltage and time dependence is given by Eqs. 5 and 6 with the appropriate parameters. Activation variables have δVₗ>0 whereas inactivation variables have δVₗ<0.

In some examples, the method may be used to perform constrained nonlinear optimization to determine values of the state variables and biological parameters that minimize the cost function (Eq. 1). The minimisation may be subject to the equality constraints fₗ(\vec{x})=0 given by the conductance model (Eq. 2) and the inequality constraints set by the search range of biological parameters and the interval of variation of the state variables. The function to minimize is the Lagrangian:

$$ℒ ( \vec{x} , λ , μ ) = c ( \vec{x} ) + {\sum }_{l = 1}^{L + 2} {λ}_{l} {f}_{l} ( \vec{x} ) - {\sum }_{l = L + 3}^{L + P + 3} {μ}_{l} ln ( {x}_{l} ) \qquad ( 9 )$$

where the λₗ are the Lagrangian multipliers associated with equality constraints and the μₗ are the inequality barriers parameters.

In some examples, a primal-dual approach is used to introduce the vector z dual to the inequality barrier μₗ, which minimizes the violation of the barrier by \vec{x}. The Lagrangian combining primal (x, λ) and dual variables (z) is:

$$ℒ ( \vec{x} , λ , μ ) = c ( \vec{x} ) + λ f ( \vec{x} ) - z \qquad ( 10 )$$

This may converted into of a linear system satisfying the Karush-Kuhn-Tucker conditions.

$$( \begin{matrix}{W}_{k} & {A}_{k} & I \\ {A}_{k}^{T} & 0 & 0 \\ {Z}_{k} & 0 & {X}_{k}\end{matrix} ) ( \begin{matrix}{d}_{k}^{x} \\ {d}_{k}^{λ} \\ {d}_{k}^{z}\end{matrix} ) = - ( \begin{matrix}∇ c ( {x}_{k} ) + {A}_{k} {λ}_{k} - {z}_{k} \\ f ( {x}_{k} ) \\ {X}_{k} {Z}_{z} e - {μ}_{j} e\end{matrix} ) \qquad ( 11 )$$

whose solution gives the search direction (dₖ^{x},dₖ^{λ},dₖ^{z}) of the (xₖ,λₖ,zₖ) at the next iteration k. Here Wₖ=∇ₓₓ² [symbol] (\vec{x},λ,z) is the Hessian of the Lagrangian; Aₖ=∇f(\vec{x}) is the Jacobian of the constraints; I is the identity matrix and e is a matrix with all is. The system in Eq. 11 can be solved iteratively generating the new state vector and Lagrangian multipliers through:

$${x}_{k + 1} = {x}_{k} + {a}_{k} {d}_{k}^{z} \qquad ( 12 )$$

$${λ}_{k + 1} = {λ}_{k} + {a}_{k} {d}_{k}^{λ}$$

$${z}_{k + 1} = {z}_{k} + {a}_{k}^{z} {d}_{k}^{z}$$

where the as specify the distance travelled in direction (dₖ^{x},dₖ^{λ},dₖ^{z}). In some examples, the system may be solved by the method of gradient descent with the interior point optimization code (IPOPT) incorporating the MA97 linear solver for sparse differential equations.

### 1.2. Equality and Inequality Constraints in Conductance Models

As mentioned above, in some examples, the optimisation is bounded by search ranges (referred to or defined as inequality constraints) for the state variables and the biological parameters. Example search ranges are defined in table 2.

### Inequality Constraints:

TABLE 2
Inequality constraints detailed for
each component of the state vector.

| Vector component of \vec{x} | Index l | Constraint |
|---|---|---|
| Membrane voltage | l = 1 | −120 mV ≤ x₁ ≤ +60 mV |
| variable | | |
| Gate variables | 2 ≤ l ≤ L | 0 ≤ xₗ ≤ 1 |
| Regularization term | l = L + P + 1 | 0 ≤ u ≤ 1 mV |
| (which may also be | | |
| referred to as a Control | | |
| variable) | | |
| Derivative of | l = L + P + 2 | −1 mV · ms⁻¹ ≤ du ≤ |
| regularization term | | 1 mV · ms⁻¹ |
| Biological Parameters | L + 1 ≤ l ≤ L + P | p_{L} ≤ pₗ ≤ p_{U} |

The choice of upper and lower bounds of the biological parameter range p_{U} and p_{L} may be guided by biological considerations. For example, the neuron membrane voltage typically oscillates between −90 mV and 45 mV and this can define the maximum possible search range for activation and inactivation voltage thresholds. As a further example, ion channel maximal conductances may typically be between 0 and 500 mS/cm². As a further example, the recovery time constants are generally commensurate with the width of action potentials (0-3 ms) for fast ion channels (Na) and the oscillations period (1-100 ms) for the other ion channels. The selected boundaries should bracket the solution. A penalty for selecting too wide a search range is a longer computation time. Otherwise, the choice of boundaries, or for that matter the initial estimates of the state variables and biological parameters within the boundaries, is independent of the eventual solution. Section 2 below demonstrates this independence as piecewise data assimilation converges towards the same biological parameter solution irrespective of the choice of the initial estimate of the state vector or the choice of the search ranges.

### Equality Constraints:

As mentioned above, constraints of the optimisation may include rate equations (Eq. 2) of the conductance model that define relationships between a state variable at a time point and the state vector at previous time points.

In some examples, the rate equations of Eq. 2 may be linearized over 3 data points (Simpson) to define the equality constraints. In some examples, the rate equations of Eq. 2 may be linearized over 5 data points (Boole) if higher accuracy is needed.
(i) Simpson's method linearizes the equations of motion over 3 points, tᵢ, tᵢ₊₁, tᵢ₊₂:

$${x}_{l} ( {t}_{i + 2} ) = {x}_{l} ( {t}_{i} ) + Δ t [ \frac{1}{3} {F}_{l} ( \vec{x} ( {t}_{i} ) ) + \frac{4}{3} {F}_{l} ( \vec{x} ( {t}_{i + 1} ) ) \frac{1}{3} {F}_{l} ( \vec{x} ( {t}_{i + 2} ) ) ] \qquad ( 13 )$$

In some examples, the mid-point at tᵢ₊₁ can be further interpolated to second order in Δt by cubic Hermite interpolation:

$${x}_{l} ( {t}_{i + 1} ) = \frac{1}{2} ( {x}_{l} ( {t}_{i} ) + {x}_{l} ( {t}_{i + 2} ) ) + \frac{Δ E}{4} [ {F}_{l} ( \vec{x} ( {t}_{i} ) ) - {F}_{l} ( \vec{x} ( {t}_{i + 2} ) ) ] \qquad ( 14 )$$

where

$$Δ t = \frac{T}{N} = {t}_{i + 1} - {t}_{i} .$$

Mid-point interpolation can advantageously avoid high frequency oscillations in the hidden state variables (the gate variables).

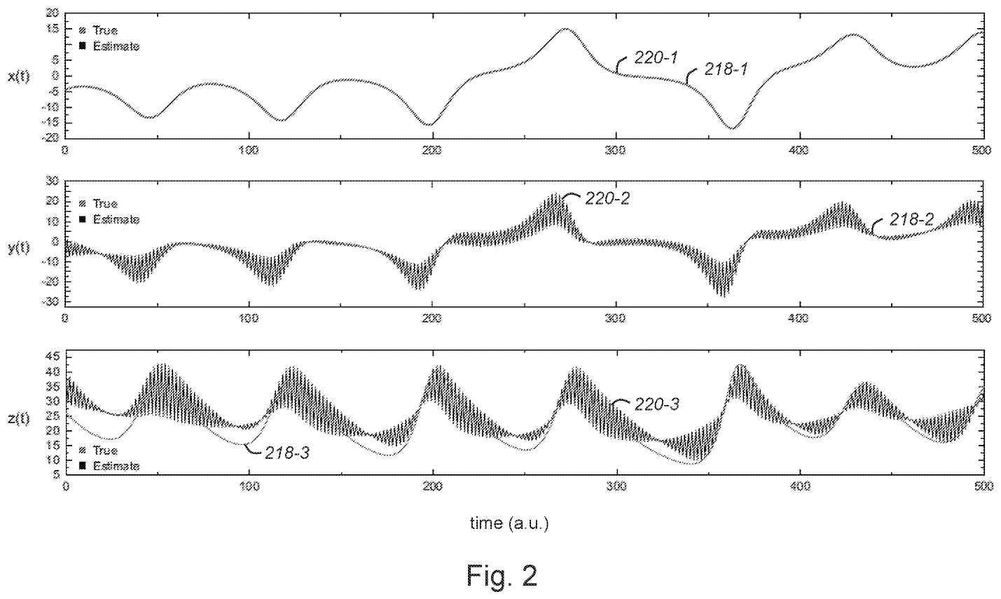

FIG. 2 shows an example Lorenz model constructed by synchronizing a variable x to data with spurious oscillations of hidden state variables y and z. The figure shows a comparison of the original Lorenz model 218-1, 218-2, 218-3 with the prediction of its twin model constructed by data assimilation 220-1, 220-2, 220-3. The data assimilation protocol omits the Hermite polynomial interpolation of the mid-point in Simpson's method (Eq. 14). Data assimilation synchronizes the state variable x(t) to the data 218-1, top panel. State variables y and z are not synchronized to their respective data in the middle and bottom panels but inferred from the synchronization of the x variable in the top panel. The absence of Hermite interpolation of the mid-point tᵢ₊₁ in the (tᵢ,tᵢ₊₁, tᵢ₊₂) interval generates a model that fits the x(t) data correctly but has incorrect parameters. As a result, the prediction of the model with these completed parameters gives spurious oscillations in the y and z variables (220-2, 220-3, middle and bottom panel).

Proof of removal of oscillations: Taylor expand at tᵢ and tₜ₊₂ and sum the two expressions retaining second order terms:

$${x}_{l} ( {t}_{i + 1} ) = {x}_{l} ( {t}_{i} ) + Δ t F ( \vec{x} ( {t}_{i} ) ) + \frac{Δ {t}^{2}}{2} {F}^{′} ( \vec{x} ( {t}_{i} ) ) +$$

$${x}_{l} ( {t}_{i + 1} ) = {x}_{l} ( {t}_{i + 2} ) - Δ t F ( \vec{x} ( {t}_{i + 2} ) ) + \frac{Δ {t}^{2}}{2} {F}^{′} ( \vec{x} ( {t}_{i + 2} ) ) +$$

$$Summing gives :$$

$$2 {x}_{i} ( {t}_{i + 1} ) = {x}_{i} ( {t}_{i} ) + {x}_{l} ( {t}_{t + 2} ) + Δ t [ F ( \vec{x} ( {t}_{i + 2} ) ) - F ( \vec{x} ( {t}_{i + 2} ) ) ] + \frac{Δ {t}^{2}}{2} [ {F}^{′} ( \vec{x} ( {t}_{i} ) ) + {F}^{′} ( \vec{x} ( {t}_{i + 2} ) ) ] +$$

Interpolating with a second order polynomial gives a constant second order derivative over the 3 points interval hence:

$${F}^{′} ( \vec{x} ( {t}_{i} ) ) = {F}^{′} ( \vec{x} ( {t}_{i + 2} ) ) \approx \frac{F ( \vec{x} ( {t}_{i + 2} ) ) - F ( \vec{x} ( {t}_{i} ) )}{2 Δ t}$$

$$Substituting :$$

$${x}_{l} ( {t}_{i + 1} ) \approx \frac{{x}_{l} ( {t}_{i} ) + {x}_{l} ( {t}_{i + 2} )}{2} + \frac{Δ t}{2} [ F ( x ( {t}_{i} ) ) - F ( x ( {t}_{i + 2} ) ) ] + \frac{Δ {t}^{2}}{4} 2 \frac{F ( \vec{x} ( {t}_{i + 2} ) ) - F ( \vec{x} ( {t}_{i} ) )}{2 Δ t}$$

$${x}_{l} ( {t}_{i + 1} ) \approx \frac{{x}_{l} ( {t}_{i} ) + {x}_{l} ( {t}_{i + 2} )}{2} + \frac{Δ t}{4} [ F ( x ( {t}_{i} ) ) - F ( x ( {t}_{i + 2} ) ) ]$$

(ii) Boole's method linearizes the equations of motion over 5 consecutive points, tᵢ, tᵢ₊₁, tᵢ₊₂, tᵢ₊₃, tᵢ₊₄:

$${x}_{l} ( {t}_{i + 4} ) = {x}_{l} ( {t}_{i} ) + 2 Δ t [ \frac{7}{45} {F}_{l} ( \vec{x} ( {t}_{i} ) ) + \frac{32}{45} {F}_{l} ( \vec{x} ( {t}_{i + 1} ) ) \frac{12}{3} {F}_{l} ( \vec{x} ( {t}_{i + 2} ) ) + \frac{32}{3} {F}_{l} ( \vec{x} ( {t}_{i + 3} ) ) \frac{32}{3} {F}_{l} ( \vec{x} ( {t}_{i + 4} ) ) ] \qquad ( 15 )$$

The 3 mid points are interpolated with:

$${x}_{l} ( {t}_{i + 1} ) = \frac{{x}_{l} ( {t}_{i} ) + {x}_{l} ( {t}_{i + 2} )}{2} + \frac{Δ t}{4} [ {F}_{l} ( x ( {t}_{i} ) ) - {F}_{l} ( x ( {t}_{i + 2} ) ) ] \qquad ( 16 )$$

$${x}_{l} ( {t}_{i + 2} ) = \frac{{x}_{l} ( {t}_{i + 1} ) + {x}_{l} ( {t}_{i + 3} )}{2} + \frac{Δ t}{4} [ {F}_{l} ( x ( {t}_{i + 1} ) ) - {F}_{l} ( x ( {t}_{i + 3} ) ) ]$$

$${x}_{l} ( {t}_{i + 3} ) = \frac{{x}_{l} ( {t}_{i + 2} ) + {x}_{l} ( {t}_{i + 4} )}{2} + \frac{Δ t}{4} [ {F}_{l} ( x ( {t}_{i + 2} ) ) - {F}_{l} ( x ( {t}_{i + 4} ) ) ]$$

In practice interpolating only two consecutive mid-points for the Boole method can prevent the spurious oscillations seen in FIG. 2. Omitting one mid-point interpolation saves significant computation time without creating spurious oscillations or reduction in accuracy. A comparison of the accuracy of the Boole and the Simpson method is shown in Table 3. As illustrated by the table, the Boole method can provide significantly better accuracy when Δt is larger.

TABLE 3
Comparing the accuracy of parameters estimated with Simpson
and Boole interpolation at different time steps Δt.
Parameters σ and ρ are parameters of the Lorenz system.

| | |
|---|---|
| Simpson's Rule [symbol] (h⁴) | Boole's Rule [symbol] (h⁶) |

| Δt | σ | ρ | σ | ρ |
|---|---|---|---|---|
| 0.005 | 5.000014 | 39.99992 | 5.000007 | 39.99996 |
| 0.01 | 5.000227 | 39.99865 | 5.000087 | 39.99952 |
| 0.02 | 5.003508 | 39.97946 | 5.001243 | 39.99378 |
| 0.04 | 5.050703 | 39.71837 | 5.015792 | 39.94637 |
| 0.05 | 5.116377 | 39.37814 | 5.032581 | 39.93155 |
| 0.06 | 5.225957 | 38.84805 | 5.053511 | 39.99929 |
| 0.07 | 5.39159 | 38.10238 | 5.068867 | 40.29379 |
| 0.08 | 5.629235 | 37.06659 | 5.053436 | 41.11519 |
| 0.09 | 5.985983 | 37.06659 | 4.943448 | 43.16304 |
| 0.1 | 6.615143 | 32.55311 | 4.574006 | 48.39547 |
| — | — | — | — | — |
| 0.16 | 10 | 22.30755 | 1.739742 | 100 |

### 1.3. Adaptive Step Size Δt

When assimilating biological data with approximate models, longer data assimilation windows (longer time) have the merit of reducing uncertainty in the biological parameter estimates. This is because statistical averaging of experimental error over more data points reduces parameter uncertainty as 1/\sqrt{N}. Secondly longer windows can increase the curvature of the cost function at the minimum giving a deeper and narrower minimum. The combination of both effects increases the accuracy on parameter solutions.

However, increasing N may result in a greater likelihood of making the Jacobian of constraints ∇f(\vec{x}) singular (Eq. 11) by introducing redundant information. This is a failure to satisfy the observability criterion. Increasing N also increases the size of the system (Eq. 11) and hence increases the computation time and rounding error.

Therefore, in some examples, the size of the assimilation window may be extended (longer duration) while keeping N constant. This trade-off is possible because most model parameters are constrained by action potential waveforms localised in time (˜2 ms) which are a small fraction of the assimilation window (˜1000 ms). A comparatively small number of parameters may control sub-threshold oscillations. Therefore, some examples may include an adaptive mesh size to sample action potentials with a finite mesh size Δt when the membrane voltage reading, Vₘₑₘ>−60 mV and sub-threshold oscillations with a mesh size mΔt when Vₘₑₘ<−60 mV. This test on the membrane voltage reading can be implemented in the example code below by setting m=1, 2, 4 depending on the value of Vₘₑₘ.

The assimilation of noisy data has shown that doubling the duration of the assimilation window can provide a ten-fold increase in parameter accuracy.

Therefore, in some examples, the method may include setting an adaptive step-size (or mesh size) of the N time points based on a value of a membrane voltage waveform. In some examples, setting an adaptive step-size may comprise: setting a finite time step, Δt, if the value of the membrane voltage waveform is greater than a threshold membrane voltage; and setting a larger time step equal to an integer multiple of the finite time step, i.e. mΔt, if the membrane voltage is less than or equal to the threshold membrane voltage. In this way, action potentials (corresponding to Vₑₘₑ>threshold membrane voltage) of the membrane voltage waveform are sampled with greater temporal resolution than sub-threshold oscillations (corresponding to Vₘₑₘ<threshold membrane voltage). As a result, the assimilation window or duration that the N time points relate to can be increased, resulting in a more accurate estimation of the biological parameters.

### 1.4. Recursive Piecewise Data Assimilation (DA)

In section 1.4.1 below, the recursive piecewise data assimilation approach is described and illustrated. In section 1.4.2, an exemplary computational implementation is described.

### 1.4.1. Recursive Piecewise DA

Conventional data assimilation evolves a conductance model continuously from the initial estimate of the state vector provided by a user (for example based on the values in Table 2). The state vector is then interpolated over every time point of the data assimilation window.

In contrast, the piecewise approach re-injects the observed membrane voltage in the model's membrane voltage variable, x₁(t), every M+1 time points while interpolating the state vector normally in between.

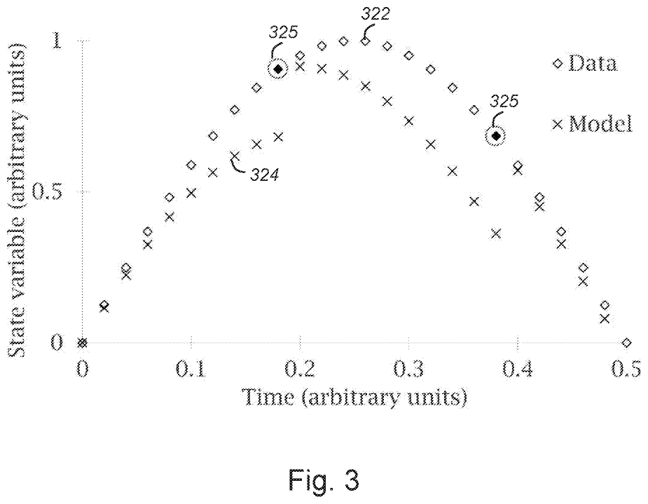

FIG. 3 illustrates an example piecewise data assimilation (DA) fitting over three segments. The values of a membrane voltage variable, x₁(t), 322 and corresponding membrane voltage readings 324 are plotted as a function of time for N time points. Each segment contains M time points. The constraints Eqs. 15 and 16 connect the neuron state (state vector values) across the M time points of each segment (M=10). At the beginning of each segment, the membrane voltage variable, x₁(t), is set equal to the observed membrane voltage reading (circled points 325). The other state variable x₂ . . . x_{L} are connected to the previous time step using Eqs 0.15 and 16. At the end of each segment, the membrane voltage state variable may be discontinuous.

The membrane voltage variable 322, is set to the membrane voltage reading 324, every 10 time points (M=10), indicated by the circled points. This periodic constraint can introduce a discontinuity in the membrane voltage variable at the end of each interval of M time points. The next model point for the membrane voltage variable in the following segment is evolved from the preceding voltage reading data point rather than the preceding model point. The unobserved state variables x₂, . . . , x_{L} may be interpolated normally across the full assimilation window.

Re-injecting data in the model (setting the membrane voltage variable to the membrane voltage reading every M time points) biases the state vector solution towards the biological solution because at every M points the observed vector components of the state vector are set to be equal to the measured data, rather than having the intermediate state depend on initial conditions. In the limit M→N this bias vanishes from the optimisation problem, except that this bias is now reflected in the initial value of the state vector. At low values of M, the membrane voltage variable, x₁(t), will present Int(N/M) discontinuities over the assimilation window of N time points. As discussed below, in some examples, a larger starting value of M may be selected if this large number of discontinuities leads to instability in the gradient descent algorithm of the parameter search (Eqs. 12 and 13). The instabilities can be resolved by reducing the bias of the model towards the data by starting from the larger value of M.

The minimum possible segment length M may correspond to the number of steps over which the equations of motion are linearised; thus 2 intervals for Simpson's method or 4 for Boole. In some examples, the segment length must always be a multiple of this minimum. A segment length M=N makes the piecewise method identical to global data assimilation. Recursive piecewise data assimilation iteratively carries out piecewise data assimilation on the same assimilation window, starting from a small segment length M=M₀ and progressively incrementing, or increasing, M in each iteration, for example until M=N.

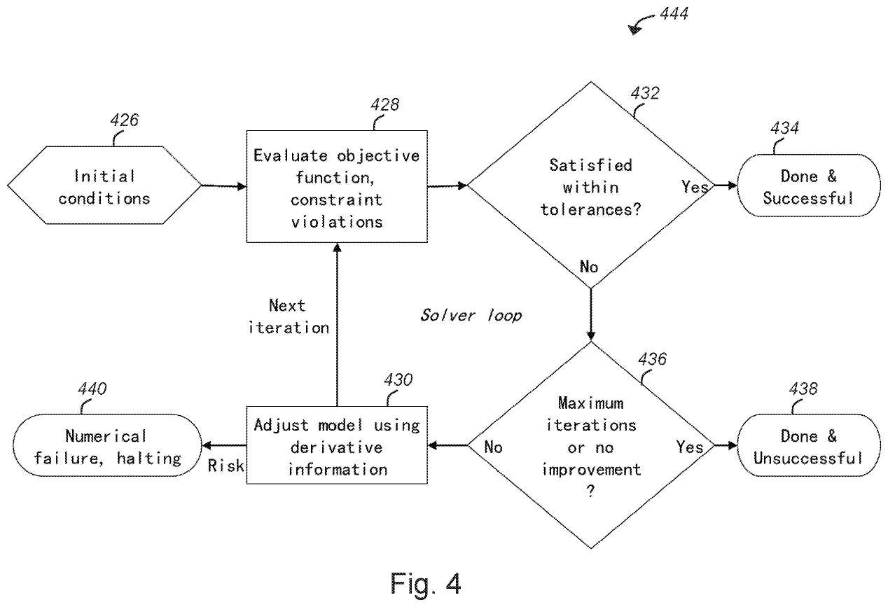

Recursive piecewise data assimilation may be implemented through the 3 algorithms illustrated in FIGS. 4 to 6. The three algorithms are nested into each other A>B>C. C is the top level and A the lower level.

FIG. 4 illustrates the lower level algorithm, A, 444 defining a generic data assimilation solver that synchronizes the neuron model to data. The DA Algorithm solver is applied to a segment of M data points. This solver minimizes the objective function under the constraints.

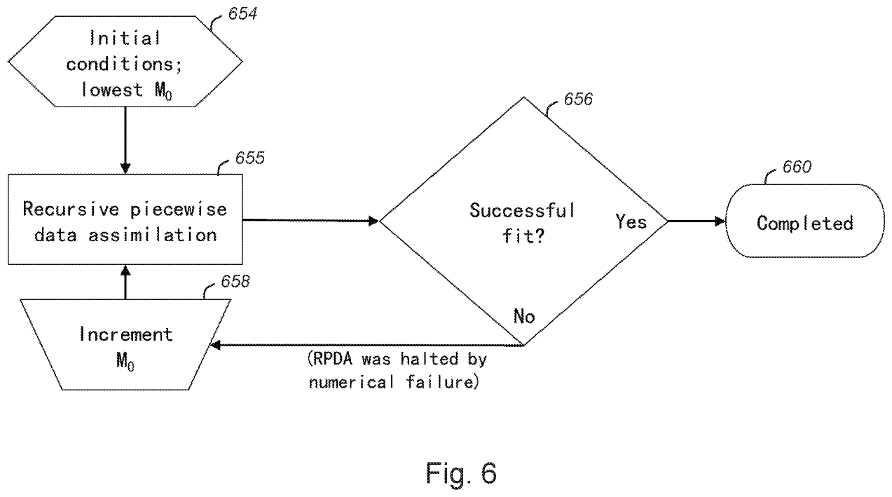

Starting from a user-provided initial estimate 426 (e.g. based on Table 2), the solver will repeatedly modify 430 the biological model parameters by iteratively solving 428 Eqs. 12 and 13. The algorithm has three possible outcomes:
If the solver can satisfy all constraints (checked at decision 432) to within the user's specified tolerances and reduce the objective function to a minimum value (for example, <10⁻⁶), it will complete successfully 434 and output the optimal set of parameters.
If it cannot do so and has exceeded a specified maximum number of iterations, or has spent multiple iterations without any improvement in constraint violations (checked at decision point 436), it will still output a parameter set but signal unsuccessful completion 438. This might indicate the solver arrived at a local minimum of the cost function which is addressed by implementing our piecewise DA method (Flowchart B)
The third possibility is that the solver may halt due to a numerical failure. For example, dealing with ill-defined systems (Eq. 11) or the discontinuities of FIG. 3 may increase memory requirements and cause the program to abort 440. This is addressed by resetting the initial value of M=M₀ in piecewise DA (see FIG. 6).

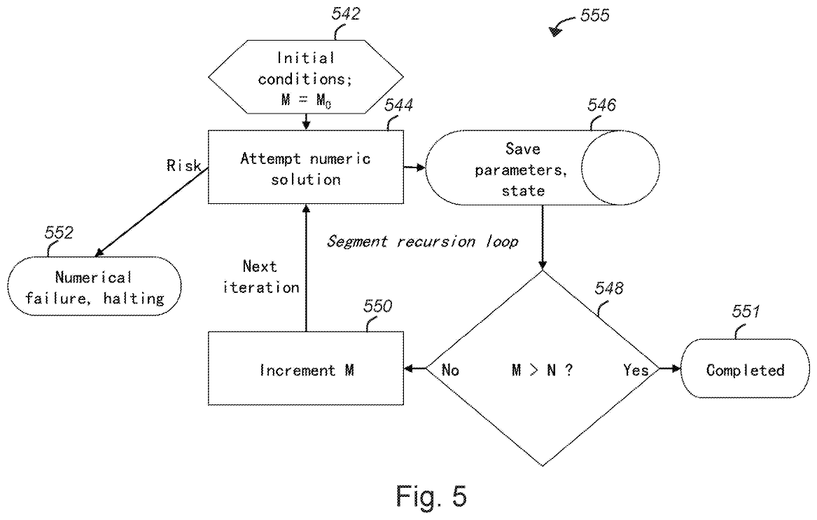

FIG. 5 illustrates the mid-level algorithm, B, of recursive piecewise data assimilation 555. Algorithm A of FIG. 4 is embedded in the “Attempt numerical solution” block 544. Algorithm B shows how the data assimilation solver of FIG. 4 may be recursively called to fit segments of length M covering the assimilation window.

The M and initial estimate of the state vector are provided at block 542. A central loop 544, 546, 548, 550 iteratively attempts a solution using algorithm A 544, saves the resulting biological parameters and state vector 546, determines if M is greater than N 548, and, if not, increases M 550, otherwise the algorithm completes 551. The purpose of the central loop is to increase the length of data segments until M=N, each time re-injecting the output parameters as input to the new parameter search. The central loop can be halted prematurely by a numeric failure 452 in the solver in which case a new increased initial value of M₀ can be provided using algorithm C, illustrated in FIG. 6.

The central loop or “segment recursion loop” sets the initial values of the biological parameters and state variables at the values output by the previous iteration of M. In this way, the successive biological parameter estimates can carry the “bias toward data” as M increases.

FIG. 6 illustrates the top-level algorithm, C, of recursive piecewise DA which includes the resetting of the initial segment size M₀ (initial value of M). Algorithm is optional and may anticipate the possibility of numerical failure arising from excessive biasing of the model towards the data.

The initial lowest value of M₀ is set at block 654 along with the initial values of the state vector. At block 655 recursive piecewise DA 655 is performed according to algorithm B. At block 656 a determination is made whether a successful fit was obtained. In the event that numerical failure halts the loop in algorithm B, algorithm C increases the initial M₀ at block 658, to the next value and begins execution of recursive piecewise DA 655 anew. This approach provides 100% convergence with a successful outcome 660 while biasing the solution towards the biologically relevant parameters.

Consider the case in which M₀ was at first set to K and in which the loop in algorithm B (FIG. 5) explored successive values of M=K, 2K, 3K, 4K . . . . If this loop fails, the outer loop in flowchart C sets M₀=2K and has the inner loop in algorithm B explore values of M=2K, 3K, 4K . . . until success or failure. Crucially, this iteration of the outer loop of algorithm C will explore different parameter and hidden state estimates at each value of M from those explored in the first execution of algorithm C. This is because the system state (value of the state vector) when starting at an initial value M₀=2K is not the same as the system state at M=2K reached in the inner loop after starting at M₀=K.

Therefore, in some examples, the method may comprise setting an initial value of M (i.e. M₀) and restarting the method with a larger initial value of M if the method terminates due to a numerical failure.

### 1.4.2. Example Implementation of Piecewise DA in Computer Code

The following description of a computer code implementation of recursive piecewise DA is merely exemplary. Other implementations may be defined without departing from the scope of the invention which is defined by the appended claims.

Key sections of the example code implementing the recursive piecewise data assimilation method are presented in the inset text below. The exemplary implementation is based on the pyDSI code (https://github.com/coin-or/Ipopt). The pyDSI code is written in Python 3 and reads information about a dynamical system in the form of a text file specifying the model equations “dyn_sys.txt” and a file specifying the starting value and search range of model parameters “bounds.txt”. It calls SymPy to perform symbolic differentiation of the Hessian of the Lagrangian (V²C) and the Jacobian of Eq. 11. pyDSI then generates C++ code expressing the dynamical system in a form compatible with an IPOPT solver, specifically using IPOPT's TNLP base class (https://coin-or.github.io/Ipopt/classIpopt_1_1TNLP.html).

The C++ code, is then compiled to create an executable. When executed, the executable reads data from two text files: one holding a time series current injected in the neuron and the other a membrane voltage time series recorded by the current clamp. IPOPT then solves the sparse linear system (Eq. 11) discretized at each point of the time window t₀ . . . t_{N}. IPOPT uses the method of gradient descent (Newton's method) to optimally synchronize the membrane voltage variable x₁(t) to the observed membrane voltage Vₘₑₘ(t). IPOPT varies the system parameters within the bounds specified by the “bounds.txt” file (for example, based on the values of table 2).

Parameters governing the behaviour of IPOPT are provided in a text file “ipopt.opt”. Parameters for problem definition, e.g. what part of the data range is to be fitted, are provided in a text file “problem_info.txt”. Parameters for the recursive piecewise DA method, such as the starting segment length and its increment, are given in a text file “mss_info.txt”. These files are read at the start of execution of the C++ executable.

### Within the C++ Code:

IPOPT TNLP class: the IPOPT TNLP is a virtual base class from which the user implements a derived class. In our case we make use of a class: class TestMSS_NLP: public TNLP which is a version of an original Test_NLP running non-piecewise DA.
A single column array of double-precision floating point numbers x stores N(L+2) double precision floating point numbers. The first N vector components hold the value of the first state variable (membrane voltage variable) across the assimilation window. The next N vector components hold the second state variable and so on, so that the first block of LN data holds the values of L state variables. The next 2*N block holds the current protocol and the voltage recorded over the data assimilation window. The last 2N block holds the control term u and its derivative du. A final block of size P holds the P parameters of the model. We can refer to this array x is as “state” of the system in general.
The number of steps M in data segments in the piecewise method is coded with integer n_mss.

The problem is defined numerically in five member functions of the class, as follows:
The objective function is defined in eval_f.
The gradient of the objective function is defined in eval_grad_f.
The constraints are defined in eval_g.
The Jacobian of the constraints is defined in eval_jac_g.
The Hessian of the Lagrangian of the problem is defined in eval_h.

These are defined as virtual functions in IPOPT's base class, and provided by the user to implement their numerical problem in a form that IPOPT can attempt to solve.

The role of the five member functions in (non-piecewise) data assimilation (DA) and how they can be modified in piecewise DA is as follows:

### The Objective Function:

The objective function (to be minimised) contains two components. The first is the sum of squares of error terms (data_[j]−x[j]) and the second is the sum of squares of values of the control variable u (here x[i+L*nObs_]), as in this instance the model included seven state variables before the control variable). In the C++ code a loop over an index variable sums all the terms contributing to this scalar over a total of nObs_ points.

```
bool TestMSS_NLP::eval_f(Index n, const Number* x,
                                  bool new_x, Number& obj_value)
{
  obj_value = 0.;
  for (Index i=0; i<nObs_; i++) {
  obj_value += (+(data_[i]-x[i])*(data_[i]-
x[i])+x[i+7*nObs_]*x[i+7*nObs_]) / 2;
  }
  return true;
}
```

The code implementing the objective function is provided as an illustration. This example objective function definition does not change in the piecewise DA approach.

### The Gradient of the Objective Function:

The gradient of the objective function is an array with the same size as x. This gradient has nonzero components with respect only to those parts of the state x that the objective explicitly depends on.

```
// return the gradient of the objective function grad_{x} f(x)
bool TestMSS_NLP::eval_grad_f(Index n, const Number* x, bool new_x,
Number* grad_f)
{
  // initialise gradient of objective function
  for (Index i=0; i<n; i++){
  grad_f[i] = 0.0;
  }
  // specify non-zero values of grad f
  for (Index i=0; i<nObs_; i++){
  grad_f[i] = (−2*data_[i] + 2*x[i]) / nObs_;
  grad_f[i+7*nObs_] = (2*x[i+7*nObs_]) / nObs_;
  }
  return true;
}
```

### the Constraints:

The equation of motion of each state variable is discretized (under Simpson interpolation) according to two equations Eq. 13 and 14. Hermite interpolation in the form of Eq. 14 is applied to the control term u to induce its smooth variation. As a result, the system has 2L+1 constraints every 2 steps of the data assimilation window.

The constraint array eval_g is a sparse (2L+1)*(N−1)/2 array storing the coefficients of the constraint equations Eq. 13 and 14. This incidentally requires the number of data points N to be an odd number, so that it will be indexed by an integer number of strides of length 2.

```
// return the value of the constraints: g(x)
bool TestMSS_NLP::eval_g(Index n, const Number* x,
                bool new_x, Index m, Number* g)
{
  Index con = 0;
  for(Index j=0; j<(nObs_-2); j+=2) {
                            if ( j % n_mss == 0 and j > 0 ){
      g[con+0*(nObs_-1)/2] = ...
      g[con+1*(nObs_-1)/2] = -0.25*h[j]*(x[j+7*nObs_]*(0.0) ...
      ...
      g[con+14*(nObs_-1)/2] = ...
                                                      }
                                                  else{
      g[con+0*(nObs_-1)/2] = ...
      g[con+1*(nObs_-1)/2] = -0.25*h[j]*(x[j+7*nObs_]*(data_[j] - x[j]) ....
      ...
      g[con+14*(nObs_-1)/2] = ...
    }
    con++;
  }
  return true;
}
```

The full equations for an exemplary system are too long to reproduce here, but the nature of the alteration made to implement the piecewise DA system can be illustrated with a code fragment as shown above.

The constraint array is constructed by a loop over an index variable j:

$$for ( Index j = 0 ; j < ( nObs\_ - 2 ) ; j += 2 )$$

The constraints themselves are indexed by a variable con which is incremented by 1 in each iteration of the loop; therefore con=j/2 in each iteration of the loop. The fifteen successive blocks of constraints are indexed by con plus an integer multiple of (N−1)/2.

The modification of the constraints occurs through a conditional which detects whether j is a multiple of the segment size, M, here written as n_mss:

$$if ( j \% n\_mss == 0 and j > 0 )$$

When this conditional is false, the constraint equations are written as usual. When the conditional is true, the constraint equations replace x[j] with data_[j] (Vₘₑₘ(tⱼ)). This implements the “restarting” of the evolution of x[j+1] and x[+2] from a data value rather than a model value. The replacement is handled in the Python code by a string substitution, illustrated here:
strings2.append(foo.replace(“x[j]”,“data_[j]”).replace(“data_[j]−data_[j]”, “0.0”))
   which writes a version of the equation in which x[j] is replaced by data_[ ]. Since j is always even in this loop construction, n_mss must also be an even number. An effect of this alteration is that when the conditional is true, the constraints are not functions of x[j].

Methods and apparatus of the present disclosure can infer changes in ion channel conductances, kinetics and/or voltage thresholds from changes in electrical activity of a cell. By linking functional changes at the cell level to changes in ion channel proteins, the disclosed methods and apparatus can identify gene mutations responsible for decay in cognitive functions. This has a consequence for the Jacobian of the constraints, as discussed below.

### The Jacobian of the Constraints

The modifications made to this function within piecewise DA are twofold. Firstly, each loop must be modified in the same way as eval_g to include a conditional based on n_mss. When the conditional is true, x[j] is replaced with data_[j] in the analytical expression of the Jacobian output by symbolic differentiation. This alteration is handled using the same string manipulation as above.

Secondly, when the conditional is true, entries corresponding to differentiation by x[j] must be zero, as these expressions are no longer functions of x[j]. This avoids the IPOPT solver attempting to satisfy a constraint that does not depend on x[j] by varying x[j].

This is implemented within the code using a Boolean filter array with one entry for each entry in the Jacobian. This filter array is set to “true” in the cases where the constraint index con, obtained from the row index by modular arithmetic, and the column index j satisfy the con=j/2 relation, and the MSS conditional is also true. Entries where the filter array is true have their values set to zero before eval_jac_g returns the value array.

The effect of this filtering is that constraints which are explicitly functions of x[j],x[j+1] and x[j+2] are differentiated by all three variables, while constraints which have become functions of data_[j],x[j+1] and x[j+2] are differentiated only by x[j+1] and x[j+2].

A C++ code fragment from the formation of the Jacobian illustrates how entries requiring this correction are identified based on their row and column indices:

```
...
  //d/dx[j] becomes zero when data_[j] replaces x[j]
  correctJacobian = new bool[nele_jac]; //array now ready
  int modulus = (nObs_-1)/2 ;//to obtain con from iRow
  int thisCon;
  for ( Index inzz = 0 ; inzz < nele_jac ; inzz++){
                                    correctJacobian[inzz] = false;
                          if (jCol[inzz] >= nObs_ or jCol[inzz] ==
0) continue; //not a d/dx[j] entry, or j=0 case
                                thisCon = iRow[inzz] % modulus;
                          if ( jCol[inzz] != 2*thisCon ) continue;
//not d/dx[j]
                          if ( jCol[inzz] % n_mss != 0 ) continue;
//not an MSS point
                          correctJacobian[inzz] = true; //a case
for treatment
                          }...
...
  for ( Index inzz = 0 ; inzz < nele_jac ; inzz++ ){
    if ( correctJacobian[inzz] ){
      values[inzz] = 0.0;
    }
  }
...
```

### the Hessian of the Lagrangian:

IPOPT also makes use of the Hessian of the Lagrangian of the problem. This entity, as discussed in the IPOPT documentation, is a matrix of second derivatives of both the objective function and the constraints. Like the Jacobian of the constraints, it is specified in a sparse matrix format by arrays recording row, column and value entries.

Like the Jacobian, the Hessian is constructed by a series of loops; the Python code writes a loop in the C++ code for each instance in which a second derivative could be nonzero. Thus the same loop modification using a conditional is made so that data_[j] is used instead of x[j] when appropriate, and so that terms whose derivative is now zero are set to zero.

The following code fragment illustrates how relevant terms in the Hessian are incremented, unless a derivative with respect to x[j] has become zero, in which case the term is not incremented; an effect achieved by commenting out the addition line.

```
...
  for (Index j=0; j<(nObs_-2); j+=2){
    inz = 0*nObs_;
    //in this case myInt is zero, so correct the Hessian for d/dx[j]
    if ( j % n_mss == 0 and j > 0 ){
//values[inz+j] += lambda[con+12*(nObs_-1)/2]*...
    inz++;
    //values[inz+j] += lambda[con+12*(nObs_-1)/2]*...
    inz++;
    //values[inz+j] += lambda[con+12*(nObs_-1)/2]*...
    inz++;
    } else {
values[inz+j] += lambda[con+12*(nObs_-1)/2]*...
    inz++;
    values[inz+j] += lambda[con+12*(nObs_-1)/2]*...
    inz++;
    values[inz+j] += lambda[con+12*(nObs_-1)/2]*...
    inz++;
      }...
```

### Iterating Over Segment Lengths to Increase the Accuracy on the Biological Solution:

The principle of iterating over gradually increasing segment lengths M has been described above (FIG. 5, Algorithm B). The purpose of this approach is to reconcile two requirements:
firstly to identify among all local minima of the cost function, the minimum that corresponds to the biological solution. A priori this is likely to be the global minimum but in ill-defined problems, this may be a local minimum.
secondly to track the solution in the vicinity of the biological minimum as the data bias is progressively removed (the biological parameter solution approaches the mathematical solution of the problem).

The iteration is achieved by a loop in the main function of the C++ code:

```
...
//loop, increasing n_mss every time (done by finalize)
  int nLoops = 0;
  while ( mynlp->n_mss < mynlp->nObsVisible ){
                      nLoops += 1;
          cout << “Attempting loop ” << nLoops << endl;
      cout << “Current n_mss ” << mynlp->n_mss << endl;
              status = app->OptimizeTNLP(mynlp);
                if (status == Solve_Succeeded) {
          printf(“\n\n*** The problem solved!\n”);
        }
        else {
          printf(“\n\n*** The problem FAILED!\n”);
        }
}...
```

The TNLP object is created once and then called repeatedly. The initial segment value is M=2. In each successive iteration, the state vector x is passed on to conserve memory of the biological minimum, except the control variable u (and du) which is reset to zero. The observed voltage state variable Vₘₑₘ(tᵢ) is set to the values in the data file, data_[i].

These alterations are handled by modifications to the constructor of our TNLP object and to its member functions get_starting_point and finalize_solution. These member functions are called automatically when IPOPT begins and ends an attempt at a numerical solution.

The constructor reads user-provided starting values of segment length M (integer) and a multiplier, which should be a real number greater than 1, from a text file, mss_info.txt. It also creates an array saved_state of size equal to the variable array x. Starting values for the state variables and parameters, obtained from a bounds.txt file, are written into this saved_state array.
The get_starting_point function is called once at the beginning of each biological parameter search. This function copies values from saved_state into the variable array x, initialising it. It then sets the first N entries of x (the voltage state (membrane voltage variable)) equal to the corresponding values in data_[i].
The finalize_solution function is a function outputting the parameters from the state vector solution x obtained by IPOPT in the format of the input “bounds.txt” file. Modification made to implement piecewise DA aimed to update the values of n_mss, x into saved_state. These will in turn be passed back into the x array at the start of the next iteration when get_starting_point is called.
Parameter values which have hit their lower or upper bound value (of the search range) are reset at the midpoint of their search range, before the next iteration.
In the exemplary implementation M is increased from one iteration to the next by multiplying the current value by a multiplier and casting the result as an integer. Two checks are then implemented. Firstly, because M must be an even number, an odd value is incremented by 1 to make it even. Secondly, if the new value is not greater than the old value, the new value becomes the old value incremented by 2. Thus M will increase by at least 2 in every cycle. A “sweep” over successive even values of M can be carried out by setting the multiplier to a value only slightly greater than 1 (e.g. 1.001), so that the increment behaviour is triggered.
The output “parameters.dat” file records the values of n_mss and the objective function as well as the parameter values themselves at the end of each iteration to evaluate the convergence of parameters).
   2. Numerical Results—Demonstration of Convergence Probability. Parameter Accuracy and Identifiability Criteria

This section describes numerical results based on an example recursive piecewise DA model and computer program according to an embodiment of the disclosure. The results illustrate how the method handles the choice of the initial piecewise interval M to achieve convergence with 100% probability. The results also illustrate that once the biological solution has been selected at smaller values of M, there is a rapid convergence over successive iterations as M: M₀→N. The results also demonstrate that piecewise data assimilation estimates parameters with very high accuracy. Parameters of the representative RVLM neuron model are estimated within 0.1% of the true parameter values used to synthesize the data. This high accuracy is maintained over 9 different assimilation windows. This demonstrates the fulfilment of identifiability criteria since the biological parameters are uniquely determined by the current protocol. The results also demonstrate that the shape of the current waveform does not affect the accuracy on parameters, as long as the current protocol probes the full dynamic range of the neuron. Finally, the results show that the estimated parameters are independent of the choice of starting conditions of the biological parameters and the state variables (the initial estimate of the state vector). The results demonstrate that piecewise data assimilation generates a unique set of biological parameter solutions, which are both accurate and physical.

Multiple criteria are used to assess convergence of the biological parameter estimation. These include:
(i) the final value of the objective function
(ii) the difference between the observed and fitted membrane voltage over the assimilation window
(iii) the value of the control variable u(t) across the window: u(t)≈0 when convergence is successful.
(iv) in the case of “twin” experiments, where the true parameter values are known, the closeness of the estimated parameters to the true parameters is used to construct model data.

These metrics are used below to demonstrate the convergence of recursive piecewise DA.

2.1. Example of Successful Convergence from a Single Starting Point M₀=2 (Algorithm B)

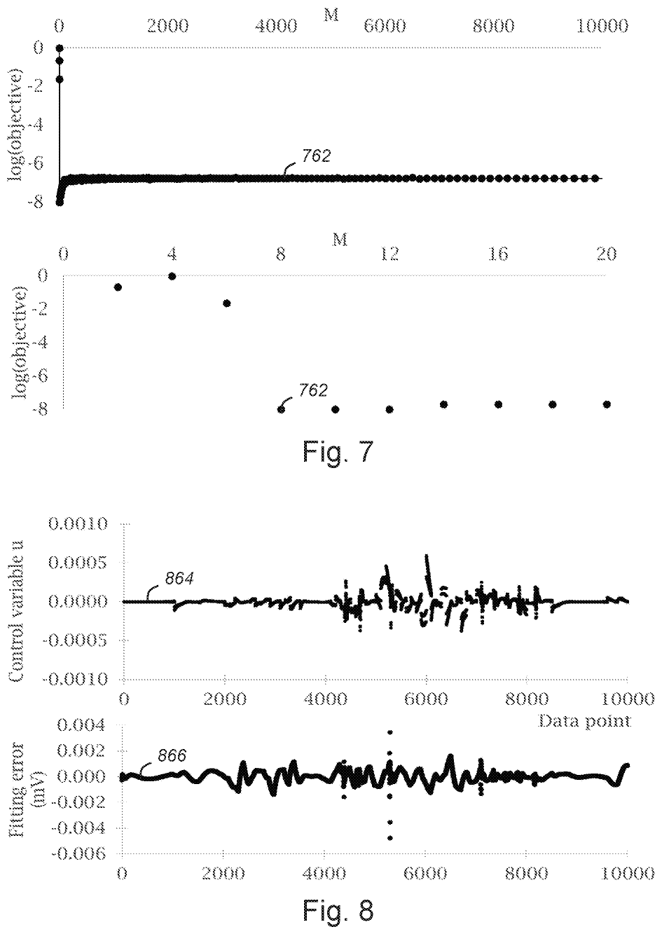

FIG. 7 illustrates the convergence of a recursive piecewise DA method according to an embodiment. In this example, PyDSI21 is executed starting from M=2 with initial gate variable and parameter values halfway between the lower bound and upper bounds of the search ranges shown in Table 2. The value of the objective function (cost function) 762 is shown as M approaches N=10,001 (top panel) and over the first few iterations M=2, . . . , 14 (bottom panel). The FIG. 7 illustrates convergence of the piecewise DA parameter search as M: 2→N. The global minimum is reached at M=8 and the value of the cost function rapidly stabilizes to less than 10⁻⁶.

FIG. 8 illustrates vanishing values of the control variable u(t) 864 and the fitting error (mismatch between model and observed membrane voltage) 866 over the assimilation window at the end of the fitting process of FIG. 7, demonstrating accuracy of the model.

2.2. Successful Convergence from Multiple Starting Points M 0=2.4.6 (Algorithm C)

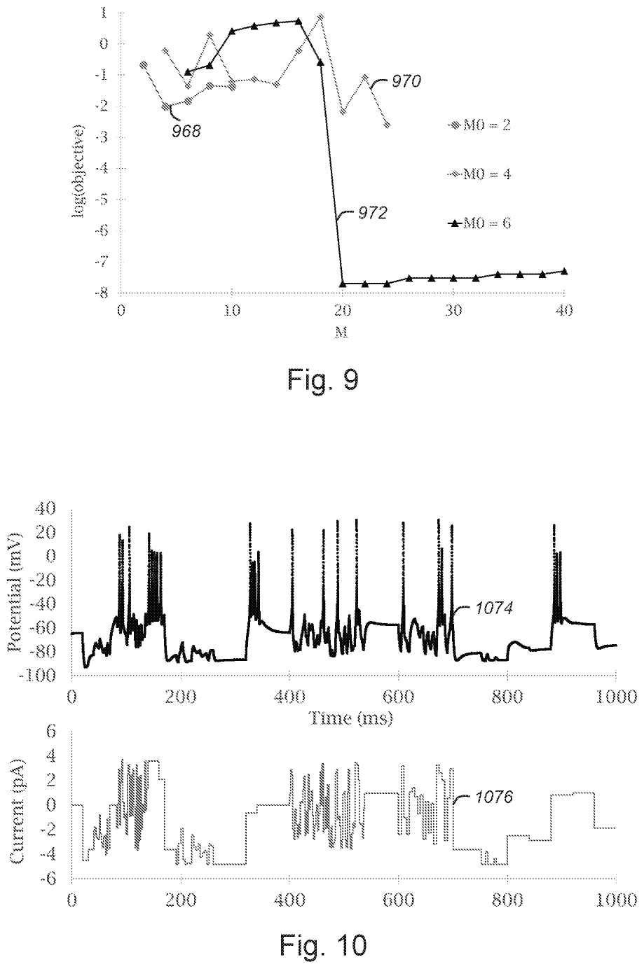

FIG. 9 illustrates convergence of recursive piecewise data assimilation starting from 3 successive values of M₀. A first sequence of iterations 968 starts at M₀=2 and halts at M=8. A second sequence of iterations 970 restarts at M₀=4 and halts at M=24. The third and final sequence of iterations 972 is initiated at M₀=6 and successfully runs until completion at M=N.

In this example, reinjection of the membrane voltage data in the model (i.e. setting the membrane voltage variable to the corresponding membrane voltage reading every M time steps) generates numerical instability at M₀=2. Therefore, Algorithm C can be implemented to restart iterations at larger values of M₀=4, 6 to reduce the bias towards the biological solution while letting mathematical optimisation proceed as naturally as possible.

### 2.3. Accuracy on Estimated Parameters (<0.1%) and Fulfilment of Identifiability Criteria

FIG. 10 illustrates model data series that was used to investigate the dependence of the biological parameters determined by piecewise DA on the waveform of the current protocol. Such analysis enables the characterization of pharmacological ion channel inhibition, as described subsequently in section 3 below. The data series includes a current protocol 1076 (bottom panel) and corresponding membrane voltage readings 1074 (top panel). Model data were generated by forward integrating the RVLM model with the current protocol 1076. The sampling rate was 50 kHz and the epoch had 50,000 data points in total. Some of the initial model parameters used to generate the model data series are shown in Table 4 (right hand column “True”). Within this epoch, 9 assimilation windows were defined each with N=10,001 data points and shifted by T=0, 5000, 10,000, . . . , 40,000 data points from the start of the epoch.

Table 4 shows representative parameter solutions of the RVLM model output by the piecewise DA method. Each column gives the biological parameters estimated from one of nine assimilation windows together with the reference parameters (right column). The biological parameters are representative of conductances g_{K}, activation voltages and widths of open to close transitions (Vₙ,dVₙ,dVₜₙ), recovery time constants (tₙϵₙ) which are particularly hard to fit, and reversal potentials (E_{K}). The estimated biological parameters in the first column deviate by less than 0.1% from the true value showing the very high accuracy of the method. The same accuracy is observed independently of the assimilation window. Hence piecewise DA estimates and uniquely identify the true parameters independently of the waveform of the current protocol. This demonstrates the identifiability condition is fulfilled across a full epoch since the accuracy of parameters remains outstanding (<0.1%) across all windows.

TABLE 4
Parameters estimated from assimilation windows of constant duration
(N = 10,001) offset by T = 0, 5000, . . . , 40,000 data points from the origin. The
maximum deviation of the estimated parameters from the true parameters (right column) is 0.1%.

| T offset | | | | | | | | | | |
|---|---|---|---|---|---|---|---|---|---|---|
| (data pts) | 0 | 5000 | 10,000 | 15,000 | 20,000 | 25,000 | 30,000 | 35,000 | 40,000 | True |
| E_{K} (mV) | −100.00 | −99.96 | −99.99 | −99.99 | −100.04 | −99.90 | −99.93 | −99.94 | −99.99 | −100 |
| g_{K} (mS · | 6.91 | 6.91 | 6.91 | 6.89 | 6.89 | 6.91 | 6.90 | 6.92 | 6.91 | 6.90 |
| cm⁻²) | | | | | | | | | | |
| Vₙ (mV) | −34.57 | −34.54 | −34.58 | −34.58 | −34.58 | −34.58 | −34.59 | −34.57 | −34.58 | −34.58 |
| dVₙ (mV) | 22.18 | 22.32 | 22.18 | 22.15 | 22.17 | 22.12 | 22.12 | 22.18 | 22.17 | 22.17 |
| dVₜₙ (mV) | 23.59 | 23.33 | 23.58 | 23.57 | 23.56 | 23.61 | 23.58 | 23.59 | 23.58 | 23.58 |
| tₙ (ms) | 1.29 | 1.30 | 1.29 | 1.29 | 1.29 | 1.29 | 1.29 | 1.29 | 1.29 | 1.29 |
| ϵₙ (ms) | 4.31 | 4.35 | 4.31 | 4.32 | 4.32 | 4.32 | 4.32 | 4.32 | 4.31 | 4.31 |

### 2.4. 100% Convergence Probability, Independent of Initial Conditions

Table 5 illustrates a study of the dependence of the biological parameter estimates on the initial estimate of the state vector (state variables and biological parameter values) at the start of piecewise data assimilation. The table shows results of the piecewise DA method for a statistical sample of 20 initial biological parameter estimates. The model used was the RVLM neuron model. The first set of 10 assimilation runs (Rand1, . . . , Rand10) initialized biological parameters at random within the search interval [p_{L},p_{U}]. The second set of 10 assimilation runs had parameters positioned uniformly at p_{L}+(p_{U}−p_{L})i/10 where j=1, . . . , 10 (Uni1, . . . , Uni10). The final series of assimilation runs used random parameters over different assimilation windows (Skip05, . . . , Skip40) as described above in relation to Table 4. The largest initial value of M corresponds to the one that led to convergence (via Algorithm C). The value of m at convergence measures the time it has taken for the parameter search to converge: cost function<10⁶ parameter error<0.10%. Table 5 illustrates a convergence rate of 100%.

TABLE 5
Searches required for the parameter search to converge to the true
parameter solutions when the initial conditions are varied.

| Initial parameters | Initial M | M at convergence |
|---|---|---|
| Rand 01 | 2 | 8 |
| Rand 02 | 2 | 24 |
| Rand 03 | 2, 4 | 8 |
| Rand 04 | 2 | 28 |
| Rand 05 | 2 | 12 |
| Rand 06 | 2 | 16 |
| Rand 07 | 2 | 14 |
| Rand 08 | 2 | 10 |
| Rand 09 | 2 | 30 |
| Rand 10 | 2, 4, 6 | 20 |
| Uni 01 | 2 | 24 |
| Uni 02 | 2 | 16 |
| Uni 03 | 2, 4 | 8 |
| Uni 04 | 2 | 28 |
| Uni 05 | 2 | 52 |
| Uni 06 | 2 | 14 |
| Uni 07 | 2 | 6 |
| Uni 08 | 2 | 16 |
| Uni 09 | 2 | 6 |
| Solution | 2 | 4 |
| Skip 05 | 4 | 4 |
| Skip 10 | 2 | 14 |
| Skip 15 | 2 | 12 |
| Skip 20 | 2 | 40 |
| Skip 25 | 2 | 112 |
| Skip 30 | 2, 4 | 16 |
| Skip 35 | 2 | 2 |
| Skip 40 | 2, 4, 6 | 76 |

In Table 5, convergence is defined as c(\vec{x})<10⁻⁶ and parameters within 0.5% of the true parameter values. The table shows that the rate of convergence depends on the choice of initial conditions (initial estimate of the state vector). However, even the slowest converging process at M=112 converges well before the final value of M=N=10,001 is reached. This demonstrates that all processes comfortably converge to the correct solution independently of the choice of the initial conditions.

This 100% probability of convergence, uniqueness of solutions and <0.1% accuracy on all parameters is an outstanding achievement of piecewise data assimilation with respect to the literature (Taylor et al., 2020, Nogaret et al., 2016; and C. D. Meliza, M. Kostuk, H. Huang, A. Nogaret, D. Margoliash, H. D. I. Abarbanel, “Estimating parameters and predicting membrane voltages with conductance-based neuron models”, Biological Cybernetics vol. 108, pp 495-516 (2014)) where convergence rate was <60%, solutions were many-fold and dependent on initial conditions.

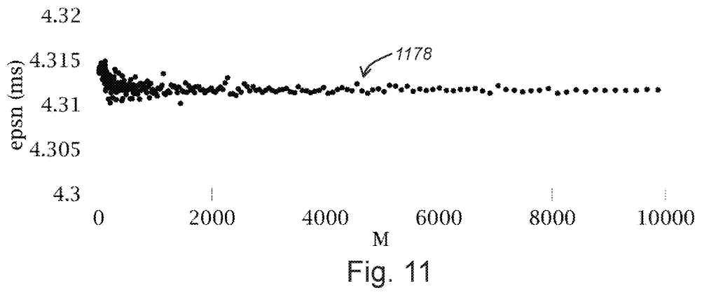

FIG. 11 shows an example convergence of a biological parameter (epsilon n intra-parameter 1178) as a function of iterations on M. This example was chosen because the time constants of conductance models are the most difficult variables to constrain and determine. This is because (in)activation time constants enter as a coefficient of an exponential time dependence of gate kinetics. The most difficult parameter to constraint is the ϵₙ intra-parameter which determines voltage dependence of the time constant. In known methods, estimation of this parameter nearly always fails as the parameter search systematically hits the boundaries of the search interval.

Remarkably, FIG. 11 shows that recursive piecewise data assimilation constrains time constant ϵₙ extremely well. The value is rapidly determined to less than 0.1% at M≈10 and the accuracy further increases as M→10,001. The true value used to generate model data was 4.314 ms.

### 2.5. Longer Assimilation Windows Increase Accuracy on Parameters

Increasing the data assimilation window from 10,001 to 15,001 data points produces essentially identical results in terms of biological parameter estimates.
Shorter assimilation windows can still give excellent accuracy on biological parameters if at least five or six action potentials are included in the time series data. A N=5,001 assimilation window spanning two or three action potentials can estimate channel conductances and reversal potentials with values similar to the original, however inaccuracies may arise in estimates of recovery time constants. An assimilation window of order 1-1.5*10⁴ points may be sufficient for obtaining maximum parameter accuracy.
Long assimilation windows can increase the number of spurious local minima in the cost function by introducing redundant information in Eq. 11 and making the Jacobian singular. However, implementing the optional adaptive step size described above in section 1.3 can address this issue.

A piecewise data assimilation method that extracts the ionic conductances, gate recovery times and activation thresholds across the full complement of ion channels by analysing the membrane voltage oscillations of an electrically active cell. The parameter solution is single valued and has the accuracy such that it can be used in a model to detect disease induced mutations in ion channel proteins or pharmacologically induced changes, as discussed below.

### 3. Application to the Identification of Pharmacologically Modified Ion Channels

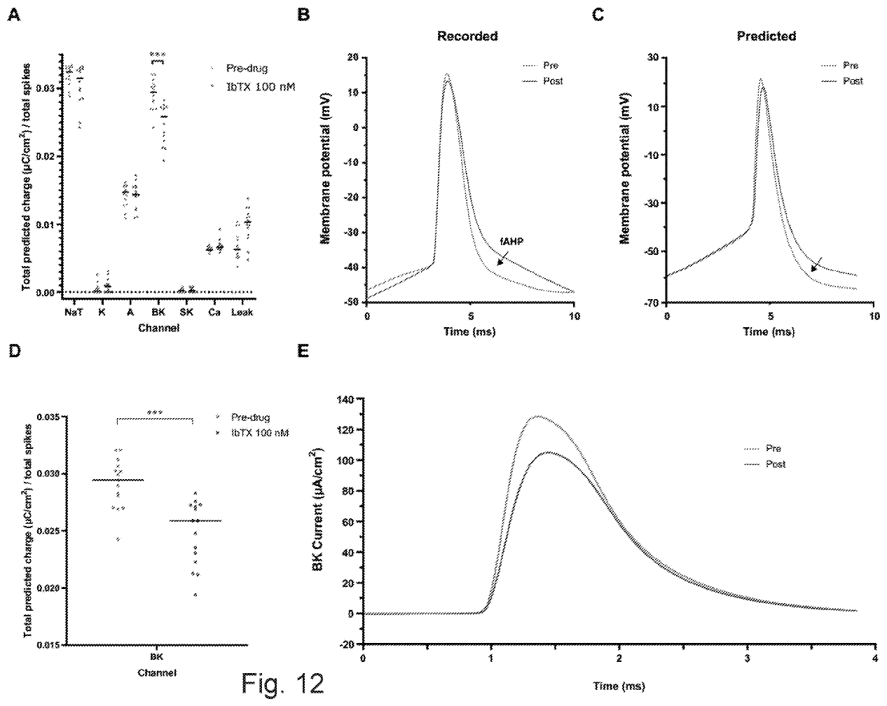

The computer-implemented method has a number of practical applications. For example, the updated state vector that is provided as a result of the method may be used to identifying whether the one or more ion-channel associated with the state vector is affected by disease based on a comparison between the updated state vector and an expected state vector. In another example, the effect of a medicament on the cell may be simulated using the updated state vector. The results of the use of the method in such applications are discussed in further detail below with reference to FIGS. 12 to 14 to provide proof of concept.

A contribution of the invention relate to the ability to provide a state vector for use in the models with sufficient accuracy to describe the cell. Once the accurate state vector is known, conventional simulation techniques can be applied to it to model changes due to drugs or disease state. In this way, data assimilation may be used in an analytical method for predicting pharmacologically-induced changes in specific ion channel currents in an excitable cell. Predictions can be made by estimating the changes in total ionic charge transferred through the cell membrane under a common stimulation protocol. As described above, the method obtains the parameters of a conductance-based neuron model that synchronise the model to observed membrane voltage oscillations. These parameters included the ionic conductance, gate recovery times and voltage thresholds of individual ion channels. Models configured with optimal parameters have been found to accurately predict the state of the neuron.

In one example, the biological parameters are determined using the method (for example using the neuron equations (e.g. Eq. 7 and 8)). These equations are then integrated using 5th order adaptive step size Runge-Kutta method or the odeint( ) routine in Python to predict the exact response of the neuron to a current stimuli. Save for the determination of the accurate biological parameters using the methods described herein, such techniques are known in the art and the skilled person may readily use alternatives. Any change in neuron oscillations due to the application of a medicament or disease) may cause very visible alterations when it is compared to the output of the digital twin (the completed neuron model) integrating the same current protocol.

In one example, the dynamics of the individual ionic currents which underly the electrical function of a hippocampal CA1 neuron were reconstructed. This had the advantage of integrating the functional overlap of individual parameters into the calculation of these currents. As a result, the uncertainty on the reconstructed current was reduced by 55% on average over the uncertainty on parameter estimates.

The method has been validated by estimating the amount of charge transferred through the neuron membrane before and after various ion channel inhibitory drugs were applied, and found good agreement between the predicted reduction in charge transferred per ion channel and the selectivity and potency of drugs for the BK, SK, A-type K⁺, and HCN currents. The selectivity of new compounds could therefore also be assessed in this way, as all ionic currents were obtained simultaneously, allowing a comparison of relative changes over all modelled channels concurrently. This approach provides a quantitative method linking changes in the electrical function of a cell to changes in specific ion channel currents. By elucidating the effect of both drug action and disease on the range of modelled ion channels, the method may enable a faster functional evaluation of drug binding, as well as revealing new targets and treatment strategies in diseases involving ion channel dysfunction.

FIG. 12 illustrates various profiles comparing recorded and predicted data regarding the medicament Iberiotoxin (IbTX), an ion channel inhibitor.

Total predicted charge across the 2000 ms prediction window for assimilations from pre-drug data (n=15), and that in 100 nM IbTX (n=15) for all channels (in FIG. 12A) and only the BK ion channel (in FIG. 12D). The data is ‘spike normalised’ by dividing by the total number of spikes across the prediction window. Horizontal lines represent median values. Asterisks represent multiplicity adjusted q values from multiple Mann-Whitney U tests using a False Discovery Rate approach (Q) of 1%. It is apparent from comparing the predicted pre- and post-drug values that the drug does not have a visible effect on the NaT, K, A-type K⁺, SK ion channels shown in FIG. 12A, but does have a noticeable effect on the BK ion channel shown in FIGS. 12A and 12D.

FIG. 12B shows an example action potential in recorded current clamp data before and during IbTX application. FIG. 12C shows averaged predicted waveforms of action potentials from all assimilations from pre-drug data, and those in IbTX. There is a qualitative similarity in the predicted and recorded values.

FIG. 12E is a magnified view of FIG. 12A focusing on the drop in ionic current through the BK channel before and after drug was applied, and represents mean predicted BK current under the same two conditions as FIGS. 12B and 12C.

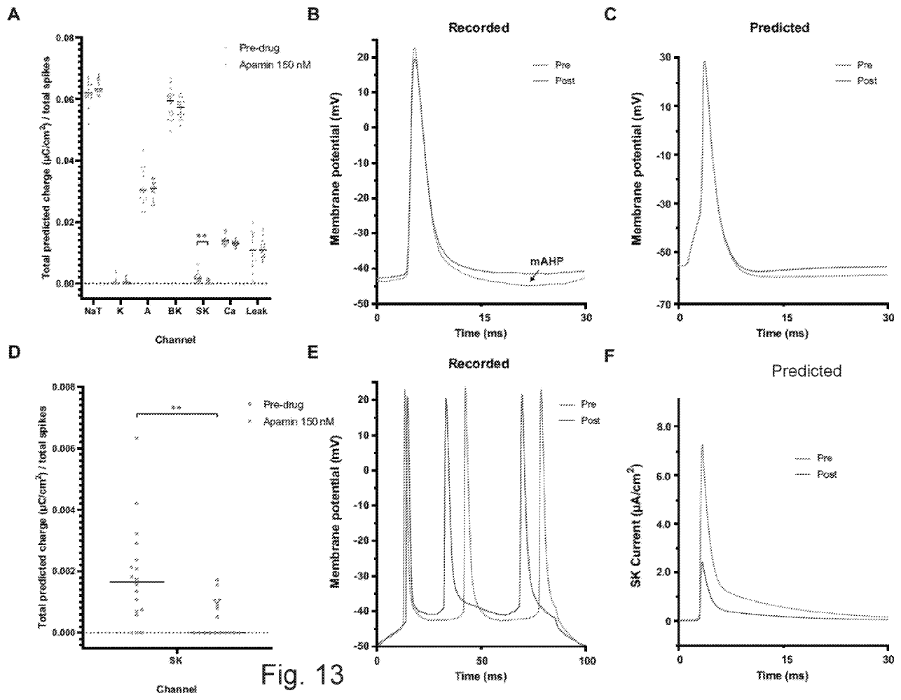

FIG. 13 illustrates various profiles comparing recorded and predicted data for cells effected by Apamin.

Total predicted charge across the 2000 ms prediction window for assimilations from pre-drug data (n=18), and that in 150 nM apamin (n=18) for all channels (in FIG. 13A) and only the SK ion channel (in FIG. 13D). As in the corresponding profiles for FIG. 12, the data is ‘spike normalised’ by dividing by the total number of spikes across the prediction window. Horizontal lines represent median values. Asterisks represent multiplicity adjusted q values from multiple Mann-Whitney U tests using a False Discovery Rate approach (Q) of 1%. It is apparent from comparing the predicted pre- and post-drug values that the drug does not have a visible effect on the NaT, K, A-type K⁺, BK and Ca ion channels shown in FIG. 13A, but does have a noticeable effect on the SK ion channel shown in FIGS. 13A and 13D.

FIGS. 13B and 13E show example action potentials in recorded current clamp data before and during apamin application. FIG. 13C shows averaged predicted waveforms of action potentials from all assimilations from pre-drug data, and those in apamin. FIG. 13F represents mean predicted SK current under these same two conditions.

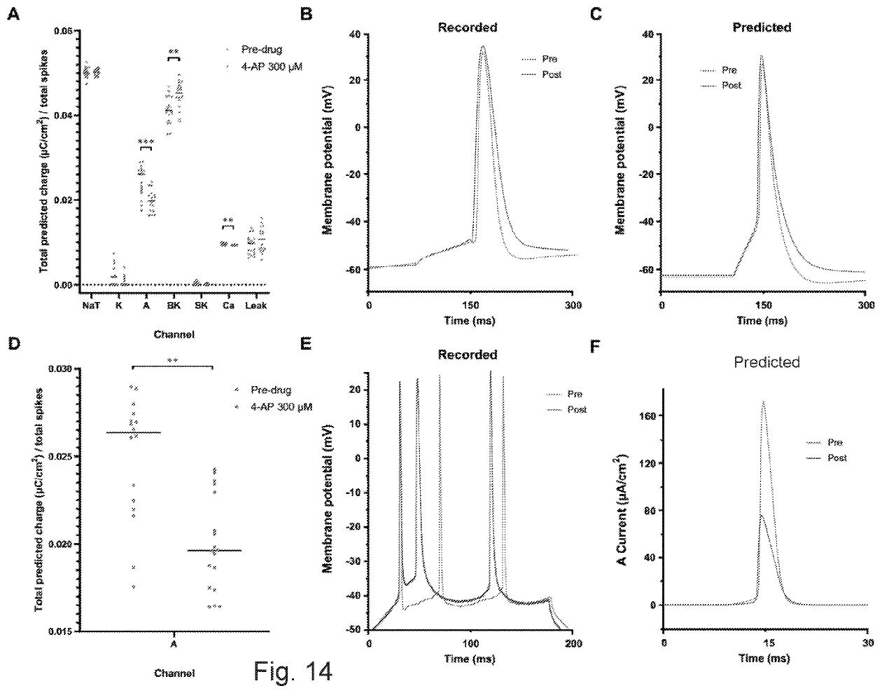

FIG. 14 illustrates various profiles comparing recorded and predicted data for cells effected by 4-aminopyridine (4-AP).

Total predicted charge across the 2000 ms prediction window for assimilations from pre-drug data (n=19), and that in 300 μM 4-AP (n=18) for all channels (in FIG. 14A) and only the A-type K⁺ ion channel (in FIG. 14D). As in the corresponding profiles for FIGS. 12 and 13, the data is ‘spike normalised’ by dividing by the total number of spikes across the prediction window. Horizontal lines represent median values. Asterisks represent multiplicity adjusted q values from multiple Mann-Whitney U tests using a False Discovery Rate approach (Q) of 1%.

FIGS. 14B and 14E show example action potentials in the recorded current clamp data before and during 4-AP application. FIG. 14C shows averaged predicted waveforms of action potentials from all assimilations from pre-drug data, and those in 4-AP. FIG. 14 F represents mean predicted A current under these same two conditions.

TABLE 5
A comparison of predicted inhibitions with literature values.

| | | | EXPECTED | | |
|---|---|---|---|---|---|
| | | | DEGREE | MEDIAN/ | RELATIVE |
| | | | OF BLOCK | MEAN | ST. DEV OF |
| | | CHANNEL | AT | PREDICTED | MEAN |
| | CONCENTRATION | TYPE | APPLIED | INHIBITION | INHIBITION |
| DRUG | APPLIED | (rodent) | CONCENTRATION | (Results) | (Results) |
| Iberiotoxin | 100 nM | BK [α | 86% | 12.1%/14.8% | 21.3% |
| | | subunit | 12% | | |
| | | only] | 77% | | |
| | | BK [α + β1] | Insensitive | | |
| | | BK [α + | [selectively | | |
| | | β3] | expressed | | |
| | | BK [α + | in HEK293 | | |
| | | β4] | cells] | | |
| Apamin | 150 nM | SK | ~100% | 100%/74.0% | 30.1% |
| | | [mAHP] | [CA1 pyr] | | |
| | | SK2 | ~100% | | |
| | | (+++) | [selectively | | |
| | | SK3 (+) | expressed | | |
| | | SK1 (++) | in xenopus | | |
| | | | oocytes] | | |
| | | | ~80% | | |
| | | | [selectively | | |
| | | | expressed | | |
| | | | in xenopus | | |
| | | | oocytes] | | |
| | | | Insensitive | | |
| | | | [selectively | | |
| | | | expressed | | |
| | | | in HEK293 | | |
| | | | cells] | | |
| 4-AP | 300 μM | A-type | ~25% | 24.3%/19.0% | 21.1% |
| | | [Kv1.4] | [selectively | | |
| | | (+) | expressed | | |
| | | A-type | in xenopus | | |
| | | [Kv4.2] | oocytes] | | |
| | | (++) | ~13% | | |
| | | | [selectively | | |
| | | | expressed | | |
| | | | in xenopus | | |
| | | | oocytes] | | |
| ZD | 50 μM | HCN | 70% [CA1 | 100%/85.5% | 24.1% |
| 7288 | | HCN1 | pyr] | | |
| | | HCN2 | 85% | | |
| | | | [DRG] | | |
| | | | ~63% | | |
| | | | [selectively | | |
| | | | expressed | | |
| | | | in HEK293 | | |
| | | | cells] | | |
| | | | ~80% | | |
| | | | [selectively | | |
| | | | expressed | | |
| | | | in xenopus | | |
| | | | oocytes] | | |

For each channel, the percentage inhibition was read from the inhibition curve at the listed drug concentration, and the citation given. The results for comparison are listed, with both median and mean predicted inhibition reported. (+)/(++)/(+++) indicates the relative amount of expression of the SK and A-type K⁺ channel types in CA1 neurons. Using our median predicted inhibition, we infer back, if possible, the drug concentration for that degree of inhibition from the inhibition curve in each study.

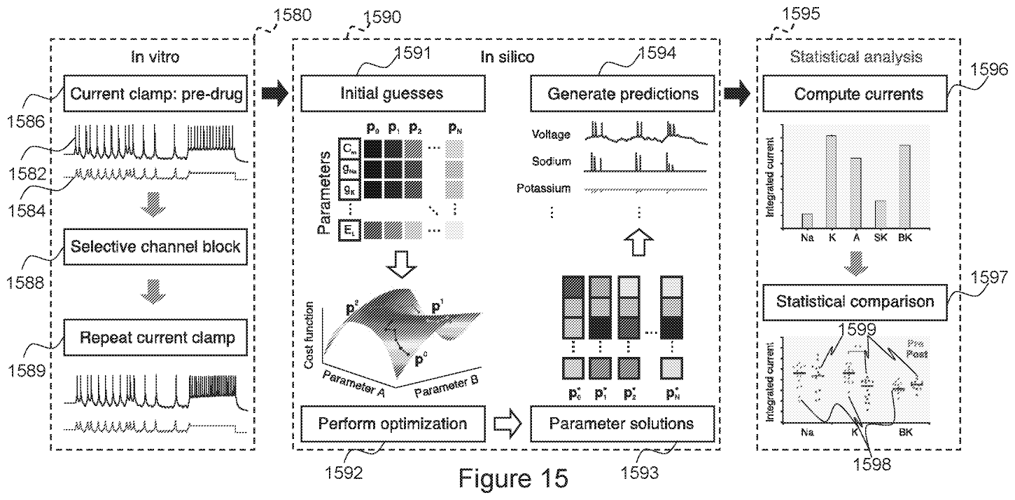

FIG. 15 illustrates a schematic diagram providing an overview of stages according to some aspects of the invention and their implementation. In this example, a proof-of-concept for inferring the potency and selectivity of ion channel blocker drugs from the electrical waveforms of CA1 neurons is provided.

In an experimental phase 1580, which may be performed in vitro or ex vitro, membrane voltage oscillations of a CA1 neuron 1582 are induced by a current protocol 1586 designed to optimally constrain ion channel parameters 1584. CA1 neuron recordings were taken before and repeated 1590 after blocking 1588 the BK channel with iberiotoxin.

In an in silico phase 1590, the assimilation method infers biological parameters {Cₘₑₘ, g_{Na}, g_{K} . . . } by synchronizing the neuron equations to membrane voltage oscillations as described above. The in silico phase comprises phases including setting an initial guess 1591 at the biological parameter values, performing an optimization procedure 1592 to provide an updated state vector solution 1593 that can be used to define the neuron to generate predictions 1594 of its behaviour.

In a statistical analysis phase 1595, the ionic charge transferred by each ion channel per action potential is calculated 1597 from the estimated parameters for ionic charge transferred in a cell before 1598 and after 1599 applying a medicament (e.g. iberiotoxin), or applying a modification due to a disease state. The method may be used to infer changes across a plurality of, or even the full complement of, ion channels in one go. Only the Na⁺ and K⁺ channels are shown for clarity in this example.

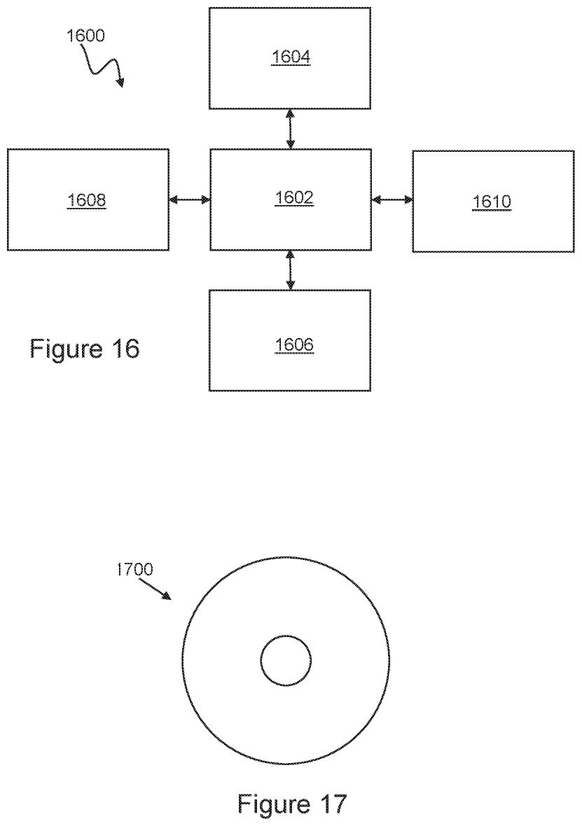

FIG. 16 illustrates a schematic block diagram of a system 1600 which may be used to implement the methods described previously. The system 1600 comprises one or more processors 1602 in communication with memory 1604. The memory 1604 is an example of a computer readable storage medium. The one or more processors 1602 are also in communication with one or more input devices 1606 and one or more output devices 1608. The various components of the system 1600 may be implemented using generic means for computing known in the art. For example, the input devices 1606 may comprise a keyboard or mouse and the output devices 1608 may comprise a monitor or display, and an audio output device such as a speaker.

In addition, the system 1600 comprises data logging hardware, which may be conventional, in order to capture measured voltage readings at least partially under the control of the one or more processors 1602. The system may be used to determine the values of biological parameters of a cell based on membrane voltage readings from a cell ex vivo using the data logging hardware.

FIG. 17 discloses a computer readable storage medium 1700. The computer readable storage medium may be used to store a computer program code configured to cause a processor to perform a method disclosed herein. In addition or alternatively, the computer readable storage medium may be used to store the data structure defining a state vector including values of biological parameters of a cell determined by the computer-implemented method.

1. A computer-implemented method for determining values of biological parameters of a cell, comprising:
receiving membrane voltage readings of the cell corresponding to N time points;
for a state vector comprising the biological parameters and a plurality of interdependent and time-dependent state variables, including a membrane voltage variable:
setting an initial guess of the state vector;
performing an optimization of the state vector by:
setting the membrane voltage variable to be equal to a corresponding membrane voltage reading at a number of time points within the N time points;
determining an updated value of the state vector by optimizing an objective function that operates on values of the membrane voltage variable and corresponding membrane voltage readings; and
repeating the optimization with:
the initial guess of the state vector set to the updated value of the state vector; and
the membrane voltage variable set to be equal to a corresponding membrane voltage reading at fewer time points within the N time points.

2. The computer-implemented method of claim 1, wherein the cell is a neuron, wherein the plurality of biological parameters relate to one or more ion channels of the neuron, and wherein the state vector is of a conductance model of the one or more ion channels of the neuron.

3. The computer-implemented method of claim 1, wherein:
performing the optimization of the state vector comprises setting the membrane voltage variable to be equal to a corresponding membrane voltage reading at intervals of M time points within the N time points, wherein M is less than N; and
repeating the optimization comprises using an increased value of M.

4. The computer-implemented method of claim 3, comprising repeating the optimization over each time point of a time window using increasing values of M until M is equal to or greater than N.

5. The computer-implemented method of claim 1, wherein the objective function includes a term based on a square of a difference between the membrane voltage variable and the corresponding membrane voltage for each time point.

6. The computer-implemented method of claim 1, wherein optimizing the objective function comprises performing a constrained non-linear optimization over each time point to determine an updated value of the state vector that minimises the objective function.

7. The computer-implemented method of claim 6, wherein constraints of the constrained non-linear optimization comprise time dependent rate equations of the state variables, wherein the rate equations define a relationship of a value of a state variable at a time point to values of the state vector at one or more previous or subsequent time points.

8. The computer-implemented method of claim 6, wherein constraints of the constrained non-linear optimization comprise search ranges comprising an upper search boundary and a lower search boundary for each state variable and each biological parameter.

9. The computer-implemented method of claim 6, wherein optimising the objective function comprises adjusting values of the plurality of biological parameters and/or state variables until the objective function satisfies a termination condition.

10. The computer-implemented method of claim 9, wherein the termination condition comprises one of: performing a maximum number of iterations of the constrained non-linear optimization; a value of function being less than a threshold; and a gradient of the objective function being less than a threshold.

11. The computer-implemented method of claim 1, wherein setting the initial guess of the state vector comprises:
setting a search range for each of the state variables and each of the biological parameters; and
setting the value of each of the state variables at each time point and each of the biological parameters as a value within a corresponding search range.

12. The computer-implemented method of claim 1, wherein the repeated optimization is performed iteratively, a plurality of times.

13. The computer-implemented method of claim 1, wherein the repeated optimization results in recursive piecewise data assimilation.

14. The computer-implemented method of claim 1, further comprising:
identifying one or more ion-channels of the cell affected by disease based on a comparison between the updated state vector and an expected state vector.

15. The computer-implemented method of claim 14, comprising identifying one or more ion-channels of the cell affected by application of a medicament based on a comparison between a state vector related to the application of the medicament and a state vector of the cell without the application of the medicament.

16. The computer-implemented method of claim 1, further comprising simulating an effect of a medicament on the cell using the updated state vector.

17. A data structure defining a state vector including values of biological parameters of a cell determined by the computer-implemented method of any of claim 1.

18. A non-transitory computer-readable storage medium comprising computer program code configured to cause one or more processors to execute the computer-implemented method of claim 1.

19. An apparatus comprising:
one or more processors; and
a memory comprising computer program code configured to cause the one or more processors to execute the computer-implemented method of claim 1.

20. The apparatus of claim 19 further comprising data logging hardware for receiving membrane voltage readings.
